(19) United States
(12) Patent Application Publication
(10) Pub. No.: US 2024/0166662 A1
(43) Pub. Date: May 23, 2024

(54) THIOPHENE BASED COMPOUNDS AND USE THEREOF AS BCKDK INHIBITORS

(71) Applicant: Ramino-Bio Ltd., Ness Ziona (IL)

(72) Inventors: Dorit COHEN CARMON, Ness Ziona (IL); Joel Moshe VAN GELDER, Jerusalem (IL)

(21) Appl. No.: 18/277,995

(22) PCT Filed: Feb. 21, 2022
(86) PCT No.: PCT/IL2022/050203

§ 371 Date: Aug. 20, 2023

Related U.S. Application Data
(60) Provisional application No. 63/151,738, filed on Feb. 21, 2021.
(60) Provisional application No. 63/280,846, filed on Nov. 18, 2021.

(51) Int. Cl. C07D 495/04; A61P 3/00

(52) U.S. Cl. CPC C07D 495/04; A61P 3/00

(57) ABSTRACT

The present invention is directed to compounds or compositions comprising thereof, and to uses thereof such as for in prevention or treatment of a disease or a disorder associated with an elevated concentration of a branched chain amino acids (BCAA) within a subject.

### CROSS REFERENCE TO RELATED APPLICATIONS

This application claims the benefit of priority under 35 USC § 119(e) of U.S. Provisional Patent Application Nos. 63/151,738, filed on Feb. 21, 2021, and 63/280,846 filed Nov. 18, 2021, the contents of which are incorporated herein by reference in their entirety.

### FIELD OF THE INVENTION

The invention relates generally to the field of compositions comprising one or more thiophene or selenophene derivatives and is directed to methods of using the same such as for treating of a disease associated with elevated concentrations of branched chain amino acids in a subject.

### BACKGROUND

Chronic elevated levels of circulating branched chain amino acids (BCAAs, such as valine, leucine, and isoleucine) are the hallmark of the orphan Maple Syrup Urine Disease (MSUD). They were recently known to be a propagating factor for cardiac hypertrophy, hepatic lipid storage, and muscle lipid accumulation, and glycemia, which contribute to various metabolic disorders.

BCAAs, three amino acids out of the nine essential amino acids, whose supply relies on food intake, are energy units but also signaling cues for protein synthesis, glucose homeostasis and metabolic signaling. BCAAs catabolic defect is a hallmark of several cardio-metabolic diseases in humans and animal models, resulting in BCAAs accumulation.

Transcriptional suppression of the BCAAs catabolic enzymes leads to decreased rates of their oxidation in visceral tissues, resulting in elevation of BCAAs levels in the blood and tissues. BCAAs accumulation contribute to the pathogenesis of cardio-metabolic diseases by activating oxidative injury and profound metabolic changes. In addition, chronic mTORC1 activation by elevated leucine levels suppresses glucose oxidation while enhanced fatty acid oxidation exacerbating lipid peroxidation toxicity.

Elevated BCAAs levels and their metabolites directly contribute to pathogenesis of insulin resistance and type 2 diabetes mellitus (T2DM) as supported by both in-vivo rodent models, integrative human genetic analyses, and human cohorts. Lowering of insulin resistance via enhanced systemic BCAA catabolism in patients with metabolic syndrome/T2DM holds potential in improving adipose and other tissue redox homeostasis, mitochondrial function, and glycaemia.

Increases in circulating BCAAs in obesity result in part from decreased rates of their oxidation in adipose tissue, due to coordinated transcriptional suppression of all the BCAA catabolic enzymes, and also from increased inactivation of the branched-chain ketoacid dehydrogenase (BCKDH) complex in liver, such that fewer BCAAs are taken up from the blood. It has been shown that BCAAs induce chronic phosphorylation of mTOR, JNK, and IRS-1, driving insulin resistance. The hypothesis is that combination of glucose, leucine, and other activators cause mTOR to start signaling for the proliferation of beta cells and the secretion of insulin. Higher concentrations of leucine cause hyperactivity in the mTOR pathway, leading to inhibition of insulin receptor substrate. It is suggested that the increased activity of mTOR complex causes eventual inability of beta cells to release insulin, and the inhibitory effect of S6 kinase leads to insulin resistance in the cells, contributing to development of type 2 diabetes.

MSUD patients having various autosomal recessive mutations in different BCAAs catabolic enzymes, has no treatment. The prevalence in a general population is 1:185,000 live births, 1:26,000 in the Ashkenazi Jewish population, and 1:358 in the Mennonite community. Dietary manipulation combined with BCAA-free synthetic formula and constant monitoring by blood and urine tests damage patients' life quality. Mental and social impairment present in most of the patients, and so far, no BCAA-reducing drug is available. MSUD is characterized by poor feeding, vomiting, lack of energy (lethargy), abnormal movements, and delayed development. If untreated, maple syrup urine disease can lead to seizures, coma, and death.

Heart Failure (HF) is regarded as an epidemic and accounts for ˜1-2% healthcare expenditure in industrialized healthcare settings. HF can be divided into 3 main categories: Chronic HF with reduced ejection fraction (HFrEF); Chronic with preserved ejection fraction (HFpEF), and acute HF (aHF). With an increasing prevalence of hypertension, obesity, atrial fibrillation, and diabetes, and the growing elderly segment of the general population, the prevalence of HFpEF is projected to increase.

While a variety of treatment strategies ranging from neurohormonal antagonists (most recently sacubitril/valsartan), devices (cardiac re-synchronization and implantable defibrillators) and cardiac transplantation exist, there are no approved drugs for HFpEF. Symptomatic metabolic modulation has long been proposed as a treatment strategy but, despite notwithstanding evidence of its pathophysiological role, has not reached frontline clinical therapy. In contrast to current drugs used, lowering BCAAs is targeting a disease-modifying mechanism that has been clinically correlated to the disease.

A possible strategy for lowering the elevated levels of circulating BCAAs, is directed to increasing catabolism of BCAAs by enhancement of branched-chain ketoacid dehydrogenase (BCKDH) complex activity. This complex controls the irreversible step of BCAAs metabolites catabolism, by oxidation of branched-chain α-keto acids (BCKAs).

BCKDH complex activity is regulated by its subunit E1α phosphorylation. BCKDH is regulated by competitive action of BCKDK kinase (a.k.a. BDK) for its inhibition by phosphorylation, and PP2Cm phosphatase for activation (see scheme below). For BCKDH activation and enhanced BCAAs catabolism, a proposed strategy is to inhibit BCKDH inhibitor, BDK. Using a synthetic BDK inhibitor tool compound, BDK inhibition enhances BCKDH activity, leading to BCAAs degradation, and lowering BCAA levels.

It is postulated that augmentation of BCAAs catabolism holds potential therapy first and foremost for orphan MSUD patients with various autosomal recessive mutations in different BCAAs catabolic enzymes. Accordingly, small molecule based specific BCKDK inhibitors are beneficial in reducing of the elevated BCAAs, by increasing catabolism thereof.

Thus, there is an unmet need to develop novel and efficient BDK inhibitors for the treatment of a disease or a disorder associated with elevated BCAAs concentration.

BCKDK was identified as a risk gene for Parkinson's disease by Genome wide association studies. BDK was identified by allelic expression profiling of genes located within PD-associated loci to identify cis-regulatory variation affecting gene expression in the non-coding region. BDK has a genetic variation in genomic loci associated with susceptibility to Parkinson's disease (PD) in both post-mortem brain tissue and whole blood samples from PD patients and controls.

The foregoing examples of the related art and limitations related therewith are intended to be illustrative and not exclusive.

### SUMMARY

The following embodiments and aspects thereof are described and illustrated in conjunction with systems, tools and methods which are meant to be exemplary and illustrative, not limiting in scope.

In one aspect of the invention, there is a compound or a salt thereof, wherein the compound is represented by or comprises Formula I:

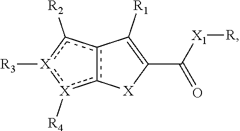

wherein: X₁ represents S or O; [symbol] represents a single or a double bond; each X is independently selected from S, Se, and CH, and at least one X is S or Se; R represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl or a combination thereof; R₃ and R₄ each independently is absent or is selected from the group comprising hydrogen, halo, optionally substituted C₁-C₆ alkyl, and C₁-C₆ haloalkyl or a combination thereof; R₁ represents hydrogen, or a substituent comprising halo, C₁-C₆ haloalkyl, or optionally substituted C₁-C₆ alkyl; R₂ represents hydrogen, or is selected from the group comprising halo, —NO₂, —CN, —OH, —CONH₂, —CONR₂, —CO₂R, —SO₂R, optionally substituted C₁-C₆ alkyl, —NH₂, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, or a combination thereof; or wherein R₂ and R₃ are interconnected so as to form a cyclic ring.

In one embodiment, the compound is represented by or comprises Formula II:

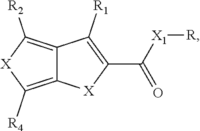

wherein at least one X is S or Se; and wherein R₄ is fluoro or a C₁-C₆ haloalkyl.

In one embodiment, R₂ is hydrogen and R₁ is selected from the group comprising hydrogen chloro, fluoro and C₁-C₆ haloalkyl.

In one embodiment, R₄ is fluoro.

In one embodiment, R₁ is fluoro or hydrogen.

In one embodiment, R is hydrogen.

In one embodiment, the compound is represented by or comprises Formula III:

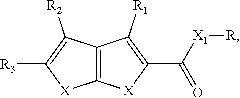

wherein at least one X is S or Se; and wherein R₃ comprises chloro, fluoro or CF₃.

In one embodiment, R₁ is fluoro, chloro or hydrogen.

In one embodiment, each of R₁ and R₃ is independently selected from hydrogen and fluoro.

In one embodiment, R₂ is selected from the group comprising hydrogen, halo, C₁-C₆ haloalkyl, NR₂, CN, OR, CONR₂, —CO₂R, SO₂R, C₁-C₆ alkyl, hydroxy(C₁-C₆ alkyl), or a combination thereof.

In one embodiment, R₂ is selected from the group comprising hydrogen, chloro, fluoro, and C₁-C₆ haloalkyl, or a combination thereof.

In one embodiment, R₂ is hydrogen.

In one embodiment, R is hydrogen.

In one embodiment, the C₁-C₆ haloalkyl is selected from the group comprising —CF₃, —CHF₂, —CH₂F, —CH₂—CF₃, —CH₂—CHF₂, —CH₂—CH₂F.

In another aspect, there is a compound or a salt thereof, wherein the compound is represented by or comprises Formula IV:

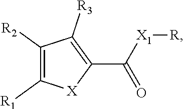

wherein X is selected from S, and Se; X₁ represents S or O; R represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl or a combination thereof; R₂ and R₃ each independently is absent or is selected from the group comprising halo, —NO₂, —CN, —OH, —CONH₂, —CONR₂, —CO₂R, —SO₂R, optionally substituted C₁-C₆ alkyl, —NH₂, —NH(C₁-C₆ alkyl), hydroxy(C₁-C₆ alkyl), C₁-C₆ haloalkyl, or a combination thereof; and R₁ represents an optionally substituted aryl, an optionally substituted heteroaryl, or R₁ and R₂ are interconnected so as to form a fused ring system.

In one embodiment, the compound is represented by or comprises Formula IVA:

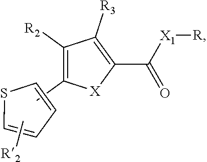

### or by Formula IVB:

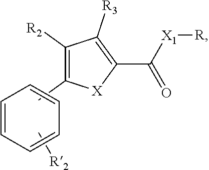

wherein X is selected from S, and Se; R represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl or a combination thereof; R₂, R′₂ and R₃ each independently is absent or is selected from the group comprising halo, —NO₂, —CN, —OH, —CONH₂, —CONR₂, —CO₂R, —SO₂R, optionally substituted C₁-C₆ alkyl, —NH₂, —NH(C₁-C₆ alkyl), hydroxy(C₁-C₆ alkyl), C₁-C₆ haloalkyl, or a combination thereof.

In one embodiment, R′₂ represents hydrogen, halo, C₁-C₆ haloalkyl, or a combination thereof.

In one embodiment, R′₂ is fluoro.

In one embodiment, the compound is represented by or comprises Formula V:

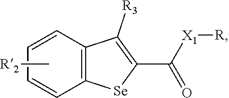

wherein R′₂ is hydrogen, or a substituent selected from the group comprising halo, —NO₂, —CN, —OH, —CONH₂, —CONR₂, —CO₂R, —SO₂R, optionally substituted C₁-C₆ alkyl, —NH₂, —NH(C₁-C₆ alkyl), hydroxy(C₁-C₆ alkyl), C₁-C₆ haloalkyl, or a combination thereof.

In one embodiment, R′₂ is halo.

In another aspect, there is a pharmaceutical composition comprising the compound of the invention, a salt thereof or both and a pharmaceutically acceptable carrier.

In one embodiment, the pharmaceutical composition comprises a therapeutically effective amount of the compound.

In one embodiment, the pharmaceutical composition is for use in the inhibition of Branched chain α-keto acid Dehydrogenase Kinase (BDK).

In one embodiment, the pharmaceutical composition is for use in prevention or treatment of a disease or a disorder associated with an elevated concentration of a branched chain amino acids (BCAA) within a subject.

In another aspect, there is a method for preventing or treating a disease or a disorder associated with elevated BCAAs concentration in a subject, comprising administering to the subject the pharmaceutical composition of the invention, thereby preventing or treating the disease or the disorder.

In one embodiment, the disease or the disorder comprises a cardiovascular disease, a metabolic disorder, a neurodegenerative disorder or any combination thereof.

In one embodiment, the cardiovascular disease comprises heart failure, congestive heart failure, acute heart failure, coronary heart disease, cardiac hypertrophy, peripheral vascular disease, renovascular disease, pulmonary hypertension, vasculitis, and acute coronary syndrome or any combination thereof.

In one embodiment, the metabolic disorder comprises maple syrup urine disease, nonalcoholic fatty liver disease, nonalcoholic steatohepatitis, hepatic lipid storage, muscle lipid accumulation, Type I diabetes, and Type II diabetes mellitus or any combination thereof.

### BRIEF DESCRIPTION OF THE DRAWINGS

FIGS. 1A-1B are bar graphs demonstrating BDK inhibitory activity of the exemplary compounds of the invention RMO-800 (FIG. 1A) and RMO-1154 (FIG. 1B) in WT and mutant lymphoblasts derived from iMSUD patients.

FIG. 2 is a bar graph demonstrating a dose dependent in-vivo reduction of BCKA (a-keto isovalerate (abbreviated as KIV), sodium a-keto isocaproate (abbreviated as KIC) and sodium a-keto β-methylvalerate (abbreviated as KMV)) concentration in the plasma of WT mice at day 8 after administration of an exemplary compound of the invention (RMO-1154). BCKA plasma concentration has been determined by LC-MS.

### DETAILED DESCRIPTION

In one aspect of the invention, there is provided a compound including any derivative and/or a salt thereof, wherein the compound is represented by or comprises Formula I:

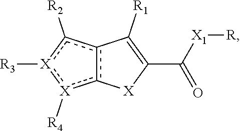

wherein: X₁ represents S, NR, or O; [symbol] represents a single or a double bond; each X is independently selected from S, Se, C and CH, and at least one X is S or Se; R represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl or a combination thereof; and if: (i) at least one X is Se, then R₂, R₃ and R₄ each independently is absent or is selected from the group comprising H, halo, —NO₂, —CN, —OH, —CONH₂, —CONR₂, —CO₂R, —SO₂R, optionally substituted C₁-C₆ alkyl, —NH₂, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, or a combination thereof; (ii) two X are S then R₁ is selected from hydrogen, F, optionally substituted C₁-C₆ alkyl, and C₁-C₆ haloalkyl or a combination thereof; R₄ and R₃ each independently are absent or selected from hydrogen, halo (e.g. F, Br, Cl, or I), optionally substituted C₁-C₆ alkyl, and C₁-C₆ haloalkyl or a combination thereof; and R₂ represents hydrogen, or is selected from the group comprising halo (e.g. F, Br, Cl, or I), —NO₂, —CN, —OH, —CONH₂, —CONR′₂, —CO₂R′, —SO₂R′, optionally substituted C₁-C₆ alkyl, —NH₂, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, or a combination thereof; wherein: at least one of R1, R2, R₃ and R₄ is not H; if at least one of R₂, R₃ and R₄ is Br, Cl, or I, then the compound comprises at least one substituent which is not Br, Cl, or I; if R₁ is F then R₂, R₃ and R₄ are not Br, Cl, or I; and if R and R₁ are not H, then at least one of R₂, R₃ and R₄ is not H; or wherein X bound to R₃ is C and R₂ and R₃ are interconnected so as to form a cyclic ring.

In some embodiments, the compound of the invention is represented by or comprises Formula IA:

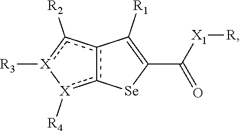

wherein X, X1, R, are as described hereinabove; [symbol] represents a single or a double bond; R₂, R₃ and R₄ each independently is absent or is selected from the group comprising H, halo, —NO₂, —CN, —OH, —CONH₂, —CONR₂, —CO₂R, —SO₂R, optionally substituted C₁-C₆ alkyl, —NH₂, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, or a combination thereof; or wherein X bound to R₃ is C and R₂ and R₃ are interconnected so as to form a cyclic ring.

In some embodiments, the compound of the invention is represented by or comprises Formula IA, wherein R₃ and R₄ each independently is absent or is selected from the group comprising hydrogen, halo, optionally substituted C₁-C₆ alkyl, and C₁-C₆ haloalkyl or a combination thereof; R₁ represents hydrogen, or a substituent comprising halo, C₁-C₆ haloalkyl, or optionally substituted C₁-C₆ alkyl; R₂ represents hydrogen, or is selected from the group comprising halo, —NO₂, —CN, —OH, —CONH₂, —CONR′₂, —CO₂R′, —SO₂R′, optionally substituted C₁-C₆ alkyl, —NH₂, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, or a combination thereof; or wherein R₂ and R₃ are interconnected so as to form a cyclic ring.

In some embodiments, the compound of the invention is represented by or comprises Formula IA1:

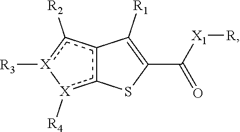

wherein: one X is S and another X is C; R₁ is selected from hydrogen, halo (e.g. F, Br, Cl, or I), optionally substituted C₁-C₆ alkyl, and C₁-C₆ haloalkyl or a combination thereof; R₄ and R₃ each independently are absent or selected from hydrogen, halo (e.g. F, Br, Cl, or I), optionally substituted C₁-C₆ alkyl, and C₁-C₆ haloalkyl or a combination thereof; and R₂ represents hydrogen, or is selected from the group comprising halo (e.g. F, Br, Cl, or I), —NO₂, —CN, —OH, —CONH₂, —CONR′₂, —CO₂R′, —SO₂R′, optionally substituted C₁-C₆ alkyl, —NH₂, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, or a combination thereof; wherein: at least one of R1, R2, R₃ and R₄ is not H; if at least one of R₂, R₃ and R₄ is Br, Cl, or I, then the compound comprises at least one substituent which is not Br, Cl, or I; if R₁ is F then R₂, R₃ and R₄ are not Br, Cl, or I and at least one of R₂, R₃ and R₄ is a substituent (wherein the substituent is as described herein); and if R and R₁ are not H, then at least one of R₂, R₃ and R₄ is not H; or wherein X bound to R₃ is C and R₂ and R₃ are interconnected so as to form a cyclic ring.

In some embodiments, the compound of the invention is represented by or comprises Formula IA2:

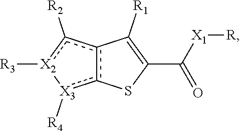

wherein X, X1, R, R1, R2, R3 and R4 are as described hereinabove (e.g. for the compounds of Formula IA1), and if X3 is S, then X2 is C, and R1, R2, R₃ and R₄ are not Br, Cl, or I. In some embodiments, the compound of the invention is represented by or comprises Formula IA2, wherein one of X2 and X3 is S and another one is C.

In some embodiments, the compound is represented by or comprises Formula IA or Formula IA1, wherein at least one X is S (e.g. one X is S and another X is C). In some embodiments, the compound is represented by or comprises Formula IA, wherein a first X is S, and a second X is C. In some embodiments, at least one X is Se. In some embodiments, the compound is represented by or comprises Formula IA or Formula IA1, wherein a first X is Se and a second X is C. In some embodiments, if X is S or Se, then any of R3 and/or R4 attached thereto is absent. In some embodiments, at least one of R2, R3 and R4 represents halo. In some embodiments, halo is F. In some embodiments, (e.g. the compound is represented by Formula IA1, wherein a first X is Se and a second X is C) R₁ is F, and at least one of R₂, R₃ and R₄ is not H, Br, Cl, or I.

In some embodiments, the compound of the invention is represented by or comprises Formula IB:

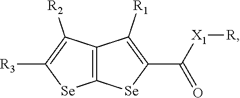

### or by Formula 1C:

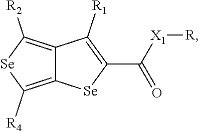

wherein X, X1, R, R1, R2, R3 and R4 are as described hereinabove (e.g. for the compounds of Formula IA).

In some embodiments, the compound of the invention is represented by or comprises Formula 1D:

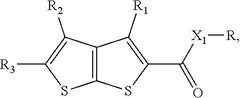

wherein X, X1, R, are as described hereinabove; R₁ is selected from hydrogen, F, optionally substituted C₁-C₆ alkyl, and C₁-C₆ haloalkyl or a combination thereof; R₄ and R₃ each independently are absent or selected from hydrogen, F, optionally substituted C₁-C₆ alkyl, and C₁-C₆ haloalkyl or a combination thereof; and R₂ represents hydrogen, or is selected from the group comprising, F, —NO₂, —CN, —OH, —CONH₂, —CONR′₂, —CO₂R′, SO₂R′, optionally substituted C₁-C₆ alkyl, —NH₂, -—NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, or a combination thereof; wherein at least one of R1, R2, R₃ and R₄ is not H. In some embodiments, the compound of the invention is represented by or comprises Formula 1D; wherein each of R1, R2, and R3 is independently H or a substituent selected from F, optionally substituted C₁-C₆ alkyl, —NR₂, —CN, —OR, —CONR₂, —CO₂R, —SO₂R, hydroxy(C₁-C₆ alkyl), and C₁-C₆ haloalkyl or a combination thereof, wherein at least one of R1, R2, and R₃ is not H. In some embodiments, the compound of the invention is represented by or comprises Formula 1D; wherein each of R1, R2, and R3 is independently H or a substituent selected from F, optionally substituted C₁-C₆ alkyl, —NR₂, —CN, —OR, —CONR₂, —CO₂R, —SO₂R, hydroxy(C₁-C₆ alkyl), and C₁-C₆ haloalkyl or a combination thereof, and wherein at least one of R1, R2, and R3 is or comprises F.

In some embodiments, the compound of the invention is represented by or comprises or by Formula 1E:

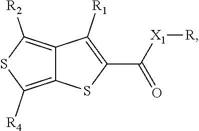

wherein X, X1, R, R1, R2, R3 and R4 are as described hereinabove (e.g. for the compounds of Formula IA2).

In some embodiments, the compound of the invention is represented by or comprises Formula II:

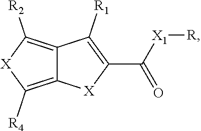

wherein X, X1, R, R1, R2, R3 and R4 are as described hereinabove, and at least one X is S or Se.

In some embodiments, at least one X is Se and R₄ comprises hydrogen, halo, C₁-C₆ alkyl and C₁-C₆ haloalkyl or a combination thereof. In some embodiments, R₄ comprises hydrogen, fluoro, chloro, and C₁-C₆ haloalkyl or a combination thereof. In some embodiments, and at least one X is S or Se and R₄ is fluoro, H, C₁-C₆ alkyl or a C₁-C₆ haloalkyl. In some embodiments, and at least one X is S or Se and R₄ is fluoro, H, or a C₁-C₆ haloalkyl. In some embodiments, and at least one X is S or Se and R₄ is fluoro, or H. In some embodiments, and at least one X is S or Se and R₄ is CFₙ, wherein n is an integer comprising 1, 2, or 3.

In some embodiments, the compound of the invention is represented by or comprises Formula IIA:

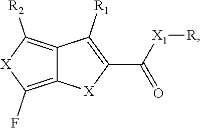

wherein X, X1, R, R1, R2, and R3 are as described hereinabove, and at least one X is S or Se. In some embodiments, any X comprises S or Se.

In some embodiments, any one of R₁ and R₂ or both is hydrogen. In some embodiments, R₂ is hydrogen and R₁ is selected from the group comprising any of hydrogen, chloro, fluoro and C₁-C₆ haloalkyl. In some embodiments, R₁ is CFₙ, wherein n is an integer comprising 1, 2, or 3. In some embodiments, R₁ is fluoro or hydrogen. In some embodiments, any one of R₁ and R₂ or both is halo. In some embodiments, any one of R₁ and R₂ or both is fluoro.

In some embodiments, the compound of the invention is represented by or comprises Formula IIB:

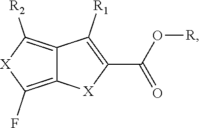

wherein X, R, R1 and R2 are as described herein.

In some embodiments, the compound of the invention is represented by or comprises Formula IIC:

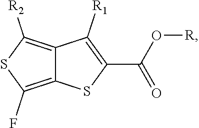

### or Formula IID:

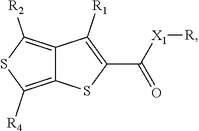

wherein X1, R, R1, R4 and R2 are as described herein. In some embodiments, the compound of the invention is represented by or comprises Formula IID, wherein: each of R1, R2, and R4 is independently H or a substituent selected from —NR′₂, —CN, —OR′, —CONR′₂, —CO₂R′, —SO₂R′, hydroxy(C₁-C₆ alkyl), F, C₁-C₆ alkyl, and C₁-C₆ haloalkyl or a combination thereof; at least one of R₁, R₂ and R₄ is not H; if R1 is F, then at least one of R2 and R4 is not H. In some embodiments, the compound of the invention is represented by or comprises Formula IID, X1, R1, R4 and R2 are as described herein, and wherein R is H or is absent. In some embodiments, the compound of the invention is represented by or comprises Formula IID, X1, R1, R4 and R2 are as described herein, and wherein R is not H, and if R1 is not H, then at least one of R2 and R4 is not H. In some embodiments, the compound of the invention is represented by or comprises Formula IID, X1, R4 and R2 are as described herein, and wherein R is not H, and R1 is H or a substituent selected from —NR′₂, —CN, —OR′, —CONR′₂, —CO₂R′, —SO₂R′, hydroxy(C₁-C₆ alkyl), C₁-C₆ alkyl, and C₁-C₆ haloalkyl or a combination thereof. In some embodiments, at least one of R1, R2, and R4 is fluoro. In some embodiments, at least one of R1, R2, and R4 is fluoro and at least one of R1, R2, and R4 is selected from an optionally substituted C₁-C₆ alkyl, and C₁-C₆ haloalkyl. In some embodiments, R1 is F and at least one of R1, R2, and R4 is selected from F, In some embodiments, at least one of R1, R2, and R4 is selected from an optionally substituted C₁-C₆ alkyl, and C₁-C₆ haloalkyl. In some embodiments, at least two of R1, R2, and R4 are fluoro.

In some embodiments, the compound of the invention is represented by or comprises Formula IIE:

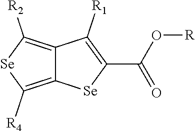

wherein R, R1, R4 and R2 are as described herein (e.g. for the compounds of Formula IA). In some embodiments, the compound of the invention is represented by or comprises Formula IIE, wherein at least one of R1, R4 and R2 is F.

In some embodiments, the compound of the invention is as described herein, wherein R₂ comprises hydrogen, halo, C₁-C₆ haloalkyl, NR₂, CN, OR, CONR₂, —CO₂R, SO₂R, C₁-C₆ alkyl, hydroxy(C₁-C₆ alkyl), or a combination thereof, wherein R is as described herein. In some embodiments, R₂ is selected from the group comprising hydrogen, chloro, fluoro, and C₁-C₆ haloalkyl, or a combination thereof.

In some embodiments, R is hydrogen. In some embodiments, the compound is represented by or comprises any one of Formulae IIC-E, and wherein R1 and/or R2 each independently comprises chloro, fluoro or hydrogen. In some embodiments, the compound is represented by or comprises any one of Formulae IIC-E, wherein R2 comprises a C₁-C₆ alkyl or C₁-C₆ haloalkyl. In some embodiments, the compound is represented by or comprises any one of Formulae IIC-E, wherein R₂ is CFₙ, wherein n is an integer comprising 1, 2, or 3. In some embodiments, the compound is represented by or comprises any one of Formulae IIC-E, wherein R₂ is fluoro or is absent. In some embodiments, the compound is represented by or comprises any one of Formulae IIC-E, wherein R₁ and R₂ are fluoro or hydrogen. In some embodiments, R₁ and R₂ are as described herein, and R is hydrogen.

In some embodiments, the compound of the invention is represented by or comprises Formula III:

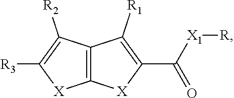

wherein at least one X is S or Se; and wherein R₁, R₂, and R₃ are as described herein.

In some embodiments, the compound of the invention is represented by or comprises Formula IIIA:

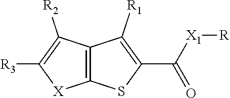

wherein X is S or Se; and wherein R_{1,} R₂, and R₃ are as described herein.

In some embodiments, the compound of the invention is represented by or comprises Formula IIIB:

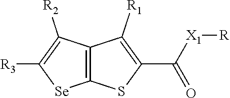

### or Formula IIIC:

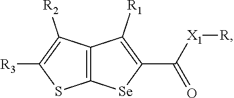

wherein R₁, R₂, and R₃ are as described herein (e.g. for the compounds of Formula IA).

In some embodiments, the compound of the invention is represented by or comprises any of Formulae IIIB-C and R₃ comprises chloro, fluoro or CFₙ, wherein n is an integer comprising 1, 2, or 3. In some embodiments, the compound of the invention is represented by or comprises any of Formulae IIIB-C and R₃ is or comprises fluoro or CF3.

In some embodiments, R₂ is hydrogen and R₁ is selected from the group comprising any of hydrogen, chloro, fluoro and C₁-C₆ haloalkyl. In some embodiments, R₁ is CFₙ, wherein n is an integer comprising 1, 2, or 3. In some embodiments, R₁ is fluoro, chloro or hydrogen. In some embodiments, any one of R₁ and R₂ or both is halo. In some embodiments, any one of R₁ and R₂ or both is fluoro. In some embodiments, any one of R₁ and R₃ is independently selected from hydrogen and halo. In some embodiments, any one of R₁ and R₃ is independently selected from hydrogen and fluoro. In some embodiments, R₁, R₂ and R₃ are selected from fluoro and chloro.

In some embodiments, the compound of the invention is as described herein, wherein R₂ comprises hydrogen, halo, C₁-C₆ haloalkyl, NR₂, CN, OR, CONR₂, —CO₂R, SO₂R, C₁-C₆ alkyl, hydroxy(C₁-C₆ alkyl), or a combination thereof, wherein R is as described herein. In some embodiments, R₂ is selected from the group comprising hydrogen, chloro, fluoro, and C₁-C₆ haloalkyl, or a combination thereof.

In some embodiments, the compound of the invention is represented by Formula IIID:

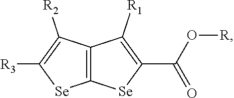

wherein R₁, R₂ and R₃ are as described herein (e.g. for the compounds of Formula IA). In some embodiments, the compound of the invention is represented by Formula IIID, wherein each of R1, R2, and R3 is independently H or a substituent selected from F, optionally substituted C₁-C₆ alkyl, —NR₂, —CN, —OR, —CONR₂, —CO₂R, —SO₂R, hydroxy(C₁-C₆ alkyl), and C₁-C₆ haloalkyl or a combination thereof and at least one of R1, R2, and R3 comprises F. In some embodiments, at least two of R₁, R₂, and R₃ are fluoro. In some embodiments, R is H.

In some embodiments, the compound of the invention is represented by Formula IIIE:

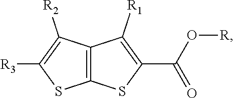

wherein R₁, R₂ and R₃ are as described herein.

In some embodiments, the compound of the invention is represented by Formula IIID-E, wherein R2 and R3 are interconnected so as to form a C5-C6 membered ring. In some embodiments, the C5-C6 membered ring is an aliphatic or an aromatic ring. In some embodiments, the C5-C6 membered ring optionally comprises one or more heteroatoms, selected form N, O and S.

In some embodiments, the compound of the invention is selected from:

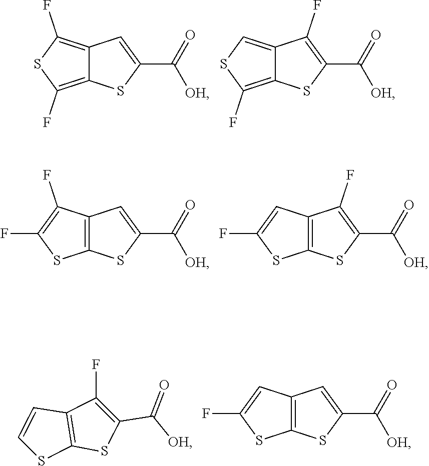

including any salt, any tautomer, any isotope, or any combination thereof.

In some embodiments, the compound of the invention is represented by Formula IIIF:

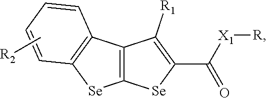

### or by Formula IIIG:

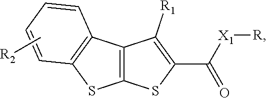

wherein R₁, R₂ and R are as described herein.

In another aspect of the invention, provided herein is a compound and/or a salt thereof, wherein the compound is represented by Formula IV:

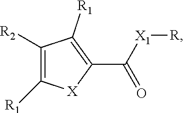

wherein X is selected from S, and Se; X₁ represents S, NR or O;
R represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl or a combination thereof; R₂ and R₃ each independently is absent or is selected from the group comprising halo, —NO₂, —CN, —OH, —CONH₂, —CONR₂, —CO₂R, —SO₂R, optionally substituted C₁-C₆ alkyl, —NH₂, —NH(C₁-C₆ alkyl), hydroxy(C₁-C₆ alkyl), C₁-C₆ haloalkyl, or a combination thereof; and R₁ represents an optionally substituted aryl, an optionally substituted heteroaryl, or R₁ and R₂ are interconnected so as to form a fused ring system.

In some embodiments, (i) R₁ and R₂ or (ii) R₂ and R₃ are interconnected so as to form a C5-C6 membered aliphatic or aromatic ring. In some embodiments, the C5-C6 membered aliphatic or aromatic ring optionally comprises one or more heteroatoms, selected form N, O and S. In some embodiments, the C5-C6 membered aliphatic or aromatic ring optionally comprises one or more substituents, wherein substituent is as described herein. In some embodiments, R₁ is devoid of tetrazole.

In some embodiments, R₁ represents an optionally substituted phenyl, an optionally substituted bicyclic aryl, or an optionally substituted C5-C6 heteroaryl, wherein substituted comprises one or more substituents as described herein.

In some embodiments, the compound of the invention is represented by Formula IVA:

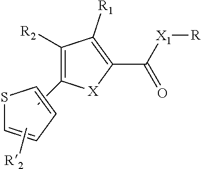

### or by Formula IVB:

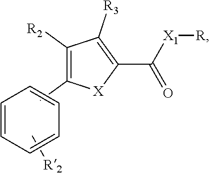

wherein X, X₁ and R are as described herein, and wherein R₂, R′₂ and R₃ each independently is absent or is selected from the group comprising halo, —NO₂, —CN, —OH, —CONH₂, —CONR₂, —CO₂R, —SO₂R, optionally substituted C₁-C₆ alkyl, —NH₂, —NH(C₁-C₆ alkyl), hydroxy(C₁-C₆ alkyl), C₁-C₆ haloalkyl, or a combination thereof.

In some embodiments, R₂ and R₃ each independently is or comprises hydrogen, C₁-C₆ haloalkyl, optionally substituted C₁-C₆ alkyl, or halo. In some embodiments, R₂ and R₃ each independently is or comprises chloro or fluoro. In some embodiments, R′₂ represents hydrogen, halo, C₁-C₆ haloalkyl, or a combination thereof. In some embodiments, R′₂ comprises fluoro or chloro. In some embodiments, X₁ is O and R is hydrogen.

In some embodiments, the compound of the invention is represented by Formula IV, wherein R₁ and R₂ are interconnected so as to form a fused ring system. In some embodiments, R₁ and R₂ are interconnected so as to form an optionally substituted aromatic ring.

In some embodiments, the compound of the invention is represented by Formula V:

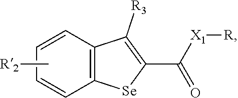

wherein X₁, R, R₃ and R′₂ are as described herein.

In some embodiments, the compound of the invention is represented by Formula V, wherein R′₂ and R₃ each independently is or comprises hydrogen, C₁-C₆ haloalkyl, optionally substituted C₁-C₆ alkyl, or halo. In some embodiments, R′₂ is halo.

In some embodiments, R′₂ and R₃ each independently is or comprises hydrogen, chloro or fluoro. In some embodiments, R′₂ represents hydrogen, halo, C₁-C₆ haloalkyl, or a combination thereof. In some embodiments, R′₂ comprises fluoro or chloro. In some embodiments, R′₂ is fluoro. In some embodiments, X₁ is O and R is hydrogen.

As used herein, the term “substituted” or the term “substituent” are related to one or more (e.g. 2, 3, 4, 5, or 6) substituents, wherein the substituent(s) is as described herein. In some embodiments, the term “substituted” or the term “substituent” comprises one or more substituents selected from (C₀-C₆)alkyl-aryl, (C₀-C₆)alkyl-heteroaryl, (C₀-C₆)alkyl-(C₃-C₈) cycloalkyl, optionally substituted C₃-C₈ heterocyclyl, halogen, NO₂, CN, OH, CONH₂, CONR₂, CNNR₂, CSNR₂, CONH—OH, CONH—NH₂, NHCOR, NHCSR, NHCNR, —NC(═O)OR, —NC(═O)NR, —NC(═S)OR, —NC(═S)NR, SO₂R, SOR, —SR, SO₂OR, SO₂N(R)₂, —NHNR₂, —NNR, C₁-C₆ haloalkyl, optionally substituted C₁-C₆ alkyl, NH₂, NH(C₁-C₆ alkyl), N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkylNR₂, C₁-C₆ alkylSR, CONH(C₁-C₆ alkyl), CON(C₁-C₆ alkyl)₂, CO₂H, CO₂R, —OCOR, —OCOR, —OC(═O)OR, —OC(═O)NR, —OC(═S)OR, —OC(═S)NR, or a combination thereof.

As used herein the term “C₁-C₆ alkyl” including any C₁-C₆ alkyl related compounds, is referred to any linear or branched alkyl chain comprising between 1 and 6, between 1 and 2, between 2 and 3, between 3 and 4, between 4 and 5, between 5 and 6, carbon atoms, including any range therebetween. In some embodiments, C₁-C₆ alkyl comprises any of methyl, ethyl, propyl, butyl, pentyl, iso-pentyl, hexyl, and tert-butyl or any combination thereof. In some embodiments, C₁-C₆ alkyl as described herein further comprises an unsaturated bond, wherein the unsaturated bond is located at 1^{st}, 2^{nd}, 3^{rd}, 4^{th}, 5^{th}, or 6^{th} position of the C₁-C₆ alkyl.

The term “(C₁-C₆) haloalkyl” describes an C₁-C₆ alkyl group as defined herein, further substituted by one or more halide(s), such as chloro, bromo and/or fluoro. In some embodiments, C₁-C₆ haloalkyl is selected from the group comprising —CX₃, —CHX₂, —CH₂X, —CH₂—CX₃, —CH₂—CHX₂, —CH₂—CH₂X, wherein X represents a halo group. In some embodiments, C₁-C₆ haloalkyl is selected from the group comprising —CF₃, —CHF₂, —CH₂F, —CH₂—CF₃, —CH₂—CHF₂, —CH₂—CH₂F.

In some embodiments, the compound of the invention comprises any one of the compounds disclosed herein, including any tautomer or an isomer thereof. In some embodiments, the compound of the invention comprises any one of the compounds disclosed herein, including any salt or any hydrate thereof. In some embodiments, the salt of the compound is a pharmaceutically acceptable salt.

### Pharmaceutical Composition

In another aspect of the invention disclosed herein, there is a pharmaceutical composition comprising a compound and/or a pharmaceutically acceptable salt thereof, wherein the compound is represented by or comprises Formula 6:

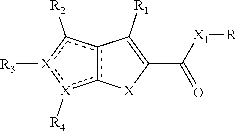

wherein:
X₁ represents S, NR′ or O; [symbol] represents a single or a double bond; each X is independently selected from S, Se, and CH, and at least one X is S or Se; R₃ and R₄ each independently is absent or is selected from the group comprising hydrogen, halo, optionally substituted C₁-C₆ alkyl, and C₁-C₆ haloalkyl or a combination thereof; R₁ represents hydrogen, or a substituent comprising halo, C₁-C₆ haloalkyl, or optionally substituted C₁-C₆ alkyl; R₂ represents hydrogen, or is selected from the group comprising halo, —NO₂, —CN, —OH, —CONH₂, —CONR′₂, —CO₂R′, —SO₂R′, optionally substituted C₁-C₆ alkyl, —NH₂, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, or a combination thereof; or wherein R₂ and R₃ are interconnected so as to form a cyclic ring; each R′ and R independently represents hydrogen, or is selected from the group comprising optionally substituted C₁-C₁₀ alkyl, optionally substituted C₃-C₁₀ cycloalkyl, optionally substituted hexose (including any dehydroxylated derivative thereof), optionally substituted pentose(including any dehydroxylated derivative thereof), optionally substituted C₃-C₁₀ heterocyclyl, optionally substituted heteroaryl, optionally substituted aryl or a combination thereof.

In some embodiments, R is absent and X1 represents a silyl group (e.g. —Si(OR′)₃), wherein each R′ independently is as described herein.

In some embodiments, at least one of R1, R2, R₃ and R₄ is not H. In some embodiments, at least two of R1, R2, R3 and R4 are not H. In some embodiments, if R and R₁ are not H, then at least one of R₂, R₃ and R₄ is not H. In some embodiments, if R and R₁ are not H, then at least one of R₂, R₃ and R₄ is halogen. In some embodiments, the compound comprises at least one, two or three halogens. In some embodiments, at least one of R1, R2, R₃ and R₄ is halogen.

In some embodiments, if at least one of R₂, R₃ and R₄ is Br, Cl, or I, then the compound comprises at least one substituent which is not Br, Cl, or I. In some embodiments, if at least one of R₂, R₃ and R₄ is Br, Cl, or I, then the compound comprises at least one F.

In some embodiments, the compound is represented by or comprises Formula 6A:

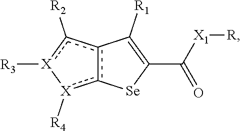

### Or Formula 6A1:

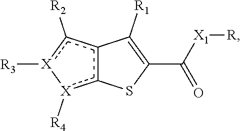

wherein X, X1, R, R1, R2, R3 and R4 are as described hereinabove; and [symbol] represents a single or a double bond. In some embodiments, the compound is represented by or comprises Formula IA or Formula IA1, wherein at least one X is S. In some embodiments, the compound is represented by or comprises Formula IA, wherein a first X is S, and a second X is C. In some embodiments, at least one X is Se. In some embodiments, the compound is represented by or comprises Formula IA or Formula IA1, wherein a first X is Se and a second X is C. In some embodiments, if X is S or Se, then any of R3 and/or R4 attached thereto is absent. In some embodiments, at least one of R3 and R4 represents halo.

In some embodiments, the compound is represented by or comprises Formula 6B:

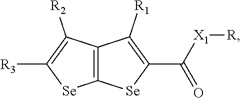

### or by Formula 6C:

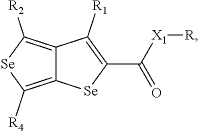

wherein X, X1, R, R1, R2, R3 and R4 are as described hereinabove.

In some embodiments, the compound is represented by or comprises Formula 6D:

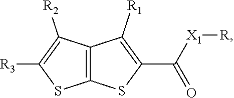

### or by Formula 6E:

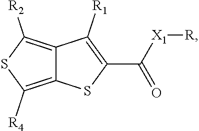

wherein X, X1, R, R1, R2, R3 and R4 are as described hereinabove.

In some embodiments, the compound is represented by or comprises Formula 6E and/or 6D, wherein at least one of R1, R2, R₃ and R₄ is not H. In some embodiments, at least two of R1, R2, R₃ and R₄ are not H. In some embodiments, if R and R₁ are not H, then at least one of R₂, R₃ and R₄ is not H. In some embodiments, if R and R₁ are not H, then at least one of R₂, R₃ and R₄ is halogen. In some embodiments, the compound comprises at least one, two or three halogens. In some embodiments, at least one of R1, R2, R₃ and R₄ is halogen.

In some embodiments, if at least one of R₂, R₃ and R₄ is Br, Cl, or I, then the compound comprises at least one substituent which is not Br, Cl, or I. In some embodiments, if at least one of R₂, R₃ and R₄ is Br, Cl, or I, then the compound comprises at least one F.

In some embodiments, the compound is represented by or comprises Formula 7:

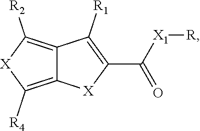

wherein X, X1, R, R1, R2, R3 and R4 are as described hereinabove, and at least one X is S or Se.

In some embodiments, R₄ comprises hydrogen, halo, C₁-C₆ alkyl and C₁-C₆ haloalkyl or a combination thereof. In some embodiments, R₄ comprises hydrogen, fluoro, chloro, and C₁-C₆ haloalkyl or a combination thereof. In some embodiments, R₄ is fluoro or a C₁-C₆ haloalkyl. In some embodiments, R₄ is CFₙ, wherein n is an integer comprising 1, 2, or 3.

In some embodiments, any one of R₁ and R₂ or both is hydrogen. In some embodiments, R₂ is hydrogen and R₁ is selected from the group comprising any of hydrogen, chloro, fluoro and C₁-C₆ haloalkyl. In some embodiments, R₁ is CFₙ, wherein n is an integer comprising 1, 2, or 3. In some embodiments, R₁ is fluoro or hydrogen. In some embodiments, any one of R₁ and R₂ or both is halo. In some embodiments, any one of R₁ and R₂ or both is fluoro.

In some embodiments, the compound is represented by or comprises Formula 7A:

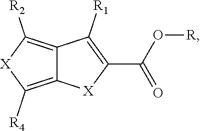

wherein X, R, R1 and R2 are as described herein.

In some embodiments, the compound is represented by or comprises Formula 7B:

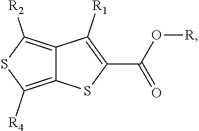

### or Formula 7C:

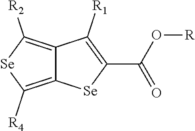

wherein R, R4, R1 and R2 are as described herein. In some embodiments, at least one of R4, R1 and R2 is or comprises halo. In some embodiments, at least one of R4, R1 and R2 is or comprises F.

In some embodiments, the compound is represented by or comprises Formulae 7B-C, wherein each of R1, R2, and R4 is independently H or a substituent selected from —NR′₂, —CN, —OR′, —CONR′₂, —CO₂R′, —SO₂R′, hydroxy(C₁-C₆ alkyl), halo (e.g. any of Br, Cl, I and/or F), C₁-C₆ alkyl, and C₁-C₆ haloalkyl or a combination thereof. In some embodiments, the compound is represented by or comprises Formulae 7B-C, wherein each of R1, R2, and R4 is independently H or halo, optionally wherein halo is F.

In some embodiments, the compound is as described herein, wherein R₂ comprises hydrogen, halo, C₁-C₆ haloalkyl, NR₂, CN, OR, CONR₂, —CO₂R, SO₂R, C₁-C₆ alkyl, hydroxy(C₁-C₆ alkyl), or a combination thereof, wherein R is as described herein. In some embodiments, R₂ is selected from the group comprising hydrogen, chloro, fluoro, and C₁-C₆ haloalkyl, or a combination thereof.

In some embodiments, R is hydrogen. In some embodiments, the compound is represented by or comprises any one of Formulae 7B-C, and wherein R1 and/or R2 each independently comprises halo (e.g. chloro and/or fluoro) or hydrogen. In some embodiments, the compound is represented by or comprises any one of Formulae 7B-C, wherein R2 comprises a C₁-C₆ alkyl or C₁-C₆ haloalkyl. In some embodiments, the compound is represented by or comprises any one of Formulae 7B-C, wherein R₂ is CFₙ, wherein n is an integer comprising 1, 2, or 3. In some embodiments, the compound is represented by or comprises any one of Formulae 7B-C, wherein R₂ is fluoro or is absent. In some embodiments, the compound is represented by or comprises any one of Formulae 7B-C, wherein R₁ and R₂ are fluoro or hydrogen. In some embodiments, R₁ and R₂ are as described herein, and R is hydrogen.

In some embodiments, the compound is represented by or comprises Formula 8:

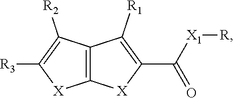

wherein at least one X is S or Se; and wherein R₁, R₂, and R₃ are as described herein.

In some embodiments, the compound is represented by or comprises Formula 8A:

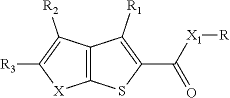

wherein X is S or Se; and wherein R₁, R₂, and R₃ are as described herein.

In some embodiments, the compound is represented by or comprises Formula 8B:

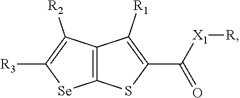

### or Formula 8C:

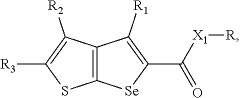

wherein R₁, R₂, and R₃ are as described herein.

In some embodiments, the compound is represented by or comprises any of Formulae 8B-C wherein each of R1, R2, and R3 is independently H or a substituent selected from halo, optionally substituted C₁-C₆ alkyl, —NR₂, —CN, —OR, —CONR₂, —CO₂R, —SO₂R, hydroxy(C₁-C₆ alkyl), and C₁-C₆ haloalkyl or a combination thereof. In some embodiments, each of R1, R2, and R3 is independently H or a substituent selected from halo, optionally substituted C₁-C₆ alkyl. In some embodiments, at least one of R1, R2, and R3 is halo. In some embodiments, at least one of R1, R2, and R3 is chloro, fluoro or CFₙ, wherein n is an integer comprising 1, 2, or 3. In some embodiments, the compound is represented by or comprises any of Formulae 8B-C and wherein two or three of R1, R2, and R3 are each independently fluoro or CF3.

In some embodiments, the compound is represented by Formula 8D:

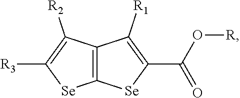

### or by Formula 8E:

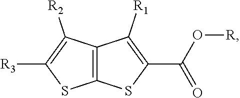

wherein R₁, R₂ and R₃ are as described herein.

In some embodiments, the compound is represented by Formula 8D-E, wherein R2 and R3 are interconnected so as to form a C5-C6 membered ring. In some embodiments, the C5-C6 membered ring is an aliphatic or an aromatic ring. In some embodiments, the C5-C6 membered ring optionally comprises one or more heteroatoms, selected form N, O and S.

In some embodiments, the compound is represented by any of Formulae 8B-E, wherein X1 is as described herein, and each of R1, R2, and R3 is independently H or a substituent selected from F, optionally substituted C₁-C₆ alkyl, —NR₂, —CN, —OR, —CONR₂, —CO₂R, —SO₂R, hydroxy(C₁-C₆ alkyl), and C₁-C₆ haloalkyl or a combination thereof; at least one X is S or Se; and at least one of R1, R2, and R4 comprises F. In some embodiments, the compound is represented by any of Formulae 8B-E, wherein R is H.

In some embodiments, the compound is or comprises any of:

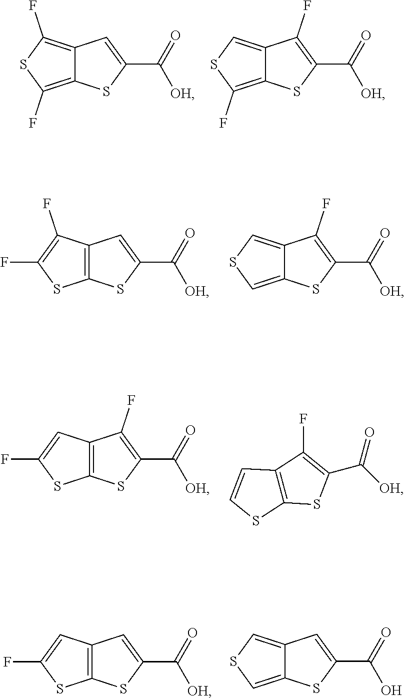

or any combination, any salt, any tautomer, or any prodrug (e.g. an aliphatic and/or aromatic ester) thereof.

In some embodiments, the compound is represented by Formula 8F:

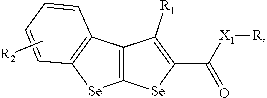

### or by Formula 8G:

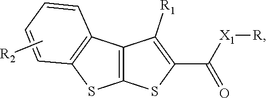

wherein R₁, R₂ and R are as described herein.

In another aspect of the invention disclosed herein, there is a pharmaceutical composition comprising the compound of the invention (e.g. any compound represented by Formulae I-V, or 1B-E) and/or a pharmaceutically acceptable salt thereof. In some embodiments, the pharmaceutical composition further comprises a pharmaceutically acceptable carrier. In some embodiments, the pharmaceutical composition comprises an effective amount of the compound of the invention and/or a pharmaceutically acceptable salt thereof. In some embodiments, the effective amount is or comprises a therapeutically effective amount.

Non-limiting examples of pharmaceutically acceptable salts include but are not limited to: acetate, aspartate, benzenesulfonate, benzoate, bicarbonate, carbonate, halide (such as bromide, chloride, iodide, fluoride), bitartrate, citrate, salicylate, stearate, succinate, sulfate, tartrate, decanoate, edetate, fumarate, gluconate, and lactate or any combination thereof.

For example, the term “pharmaceutically acceptable” can mean approved by a regulatory agency of the Federal or a state government or listed in the U.S. Pharmacopeia or other generally recognized pharmacopeia for use in animals, and more particularly in humans. In some embodiments, the compound of the invention is referred to herein as an active ingredient of a pharmaceutical composition.

In some embodiments, the pharmaceutical composition as described herein is a topical composition. In some embodiments, the pharmaceutical composition is an oral composition. In some embodiments, the pharmaceutical composition is an injectable composition. In some embodiments, the pharmaceutical composition is for a systemic use.

In some embodiments, the pharmaceutical composition is any of an emulsion, a liquid solution, a gel, a paste, a suspension, a dispersion, an ointment, a cream, or a foam.

As used herein, the term “carrier” refers to a diluent, adjuvant, excipient, or vehicle with which the active ingredient is administered. Such carriers can be sterile liquids, such as water-based and oils, including those of petroleum, animal, vegetable, or synthetic origin, such as peanut oil, soybean oil, mineral oil, sesame oil and the like, polyethylene glycols, glycerin, propylene glycol or other synthetic solvents.

Other non-limiting examples of carriers include, but are not limited to: terpenes derived from Cannabis, or total terpene extract from Cannabis plants, terpenes from coffee or cocoa, mint-extract, eucalyptus-extract, citrus-extract, tobacco-extract, anis-extract, any vegetable oil, peppermint oil, d-limonene, b-myrcene, a-pinene, linalool, anethole, α-bisabolol, camphor, β-caryophyllene and caryophyllene oxide, 1,8-cineole, citral, citronella, delta-3-carene, farnesol, geraniol, indomethacin, isopulegol, linalool, unalyl acetate, β-myrcene, myrcenol, 1-menthol, menthone, menthol and neomenthol, oridonin, a-pinene, diclofenac, nepafenac, bromfenac, phytol, terpineol, terpinen-4-ol, thymol, and thymoquinone. One skilled in the art will appreciate, that a particular carrier used within the pharmaceutical composition of the invention may vary depending on the route of administration.

In some embodiments, the carrier improves the stability of the active ingredient in a living organism. In some embodiments, the carrier improves the stability of the active ingredient within the pharmaceutical composition. In some embodiments, the carrier enhances the bioavailability of the active ingredient.

Water may be used as a carrier such as when the active ingredient has a sufficient aqueous solubility, so as to be administered intravenously. Saline solutions and aqueous dextrose and glycerol solutions can also be employed as liquid carriers, particularly for injectable solutions.

In some embodiments, the carrier is a liquid carrier. In some embodiments, the carrier is an aqueous carrier.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The composition, if desired, can also contain minor amounts of wetting or emulsifying agents, or pH buffering agents such as acetates, citrates, or phosphates. Antibacterial agents such as benzyl alcohol or methyl parabens; antioxidants such as ascorbic acid or sodium bisulfate; and agents for the adjustment of tonicity such as sodium chloride or dextrose are also envisioned. The carrier may comprise, in total, from 0.1% to 99.99999% by weight of the composition/s or the pharmaceutical composition/s presented herein.

In some embodiments, the pharmaceutical composition includes incorporation of any one of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions may influence the physical state, solubility, stability, rate of in vivo release, and rate of in vivo clearance.

In some embodiments, the pharmaceutical composition is a liquid at a temperature between 15 to 45° C. In some embodiments, the pharmaceutical composition is a solid at a temperature between 15 to 45° C. In some embodiments, the pharmaceutical composition is a semi-liquid at a temperature between 15 to 45° C. It should be understood that the term “semi-liquid”, is intended to mean materials which are flowable under pressure and/or shear force. In some embodiments, semi-liquid compositions include creams, ointments, gel-like materials, and other similar materials. In some embodiments, the pharmaceutical composition is a semi-liquid composition, characterized by a viscosity in a range from 31,000-800,000 cps.

Non-limiting examples of carriers for pharmaceutical compositions being in the form of a cream include but are not limited to: non-ionic surfactants (e.g., glyceryl monolinoleate glyceryl monooleate, glyceryl monostearate lanolin alcohols, lecithin mono- and di-glycerides poloxamer polyoxyethylene 50 stearate, and sorbitan trioleate stearic acid), anionic surfactants (e.g. pharmaceutically acceptable salts of fatty acids such as stearic, oleic, palmitic, and lauric acids), cationic surfactants (e.g. pharmaceutically acceptable quaternary ammonium salts such as benzalkonium chloride, benzethonium chloride, and cetylpyridinium chloride) or any combination thereof.

In some embodiments, the pharmaceutical composition being in the form of a cream further comprises a thickener.

Non-limiting examples of thickeners include, but are not limited to microcrystalline cellulose, a starch, a modified starch, gum tragacanth, gelatin, and a polymeric thickener (e.g. polyvinylpyrrolidone) or any combination thereof.

In some embodiments, the pharmaceutical composition comprising the compound of the invention is in a unit dosage form. In some embodiments, the pharmaceutical composition is prepared by any of the methods well known in the art of pharmacy. In some embodiments, the unit dosage form is in the form of a tablet, capsule, lozenge, wafer, patch, ampoule, vial, or pre-filled syringe.

In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems. In some embodiments, the effective dose is determined as described hereinabove.

In another embodiment, the pharmaceutical composition of the invention is administered in any conventional oral, parenteral, or transdermal dosage form.

As used herein, the terms “administering”, “administration”, and like terms refer to any method which, in sound medical practice, delivers a composition containing an active agent to a subject in such a manner as to provide a therapeutic effect.

In some embodiments, the pharmaceutical composition is administered via oral (i.e., enteral), rectal, vaginal, topical, sublingual, buccal, nasal, ophthalmic, transdermal, subcutaneous, intramuscular, intraperitoneal, intrathecal, or intravenous routes of administration. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. In addition, it may be desirable to introduce the pharmaceutical composition of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

In some embodiments, the pharmaceutical composition is in a form of, for example, and not by way of limitation, an ointment, cream, gel, paste, foam, aerosol, suppository, pad, or gelled stick.

In some embodiments, for oral applications, the pharmaceutical composition is in the form of a tablet or a capsule, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. In some embodiments, the tablet of the invention is further film coated. In some embodiments, oral application of the pharmaceutical composition or of the kit is in a form of a drinkable liquid. In some embodiments, oral application of the pharmaceutical composition or of the kit is in a form of an edible product.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous, and intraperitoneal injection purposes.

In some embodiments, the pharmaceutical composition of the invention is for use in the inhibition of Branched chain α-keto acid Dehydrogenase Kinase (BDK). In some embodiments, inhibition of BDK comprises inhibition of cellular activity of BDK, wherein inhibition is as described herein. In some embodiments, inhibition comprises a selective inhibition of BDK activity. In some embodiments, the pharmaceutical composition or the compound of the invention is for use in the inhibition of BDK activity within at least one cell of the subject. In some embodiments, the cell expresses at least one PP2Cm mutant. In some embodiments, the cell expresses a wild-type PP2Cm.

In some embodiments, the pharmaceutical composition of the invention is for use in enhancing BCKDH activity. In some embodiments, enhancing BCKDH activity is by inhibition of BDK activity.

In some embodiments, the compound of the invention is characterized by a selective inhibition of BDK activity at an allosteric pocket thereof

In some embodiments, the compound has at least 2 times, at least 5 times, at least 10 times, at least 15 times, at least 20 times, at least 30 times, at least 30 times, at least 50 times, at least 80 times, at least 100 times, at least 200 times, at least 300 times, at least 400 times, at least 500 times, at least 700 times, at least 1000 times, at least 10,000 times, at least 50,000 times, at least 100,000 times higher binding affinity for the allosteric pocket of BDK as compared to the ATP binding site of the BDK.

In some embodiments, the pharmaceutical composition of the invention is for use in the reduction of a branched-chain amino acid(s) (BCAA) concentration within at least one cell a subject. In some embodiments, the pharmaceutical composition of the invention is for use in the prevention or reduction of BCAA concentration within a tissue and/or blood of a subject.

In some embodiments, the pharmaceutical composition of the invention is for use in the enhancement BCAA catabolism. In some embodiments, the pharmaceutical composition of the invention is for use in the enhancement activity of branched-chain ketoacid dehydrogenase (BCKDH) complex.

In some embodiments, the pharmaceutical composition of the invention is for use in prevention or treatment of a disease or a disorder associated with an elevated concentration of a branched chain amino acids (BCAA) within a subject (e.g. within a tissue and/or blood). In some embodiments, the disease or the disorder comprises a cardiovascular disease, a metabolic disorder or both.

In some embodiments, the pharmaceutical composition of the invention is for use in prevention or treatment of a disease or a disorder selected from heart failure, congestive heart failure, acute heart failure, coronary heart disease, cardiac hypertrophy, peripheral vascular disease, renovascular disease, pulmonary hypertension, vasculitis, acute coronary syndrome maple syrup urine disease, nonalcoholic fatty liver disease, nonalcoholic steatohepatitis, hepatic lipid storage, muscle lipid accumulation, Insulin resistance, Type I diabetes, and Type II diabetes mellitus or any combination thereof.

### Method

In another aspect, there provided herein is a method for preventing or treating a disease or a disorder, or ameliorating a condition associated with an abnormal or normal BCAAs concentration within a subject, the method comprises administering to the subject an effective amount of the pharmaceutical composition or of the compound of the invention, thereby (i) preventing or treating the disease and/or the disorder; or (ii) ameliorating the condition associated with an abnormal or normal BCAAs concentration within the subject.

In another aspect, there provided herein is a method for preventing or treating a disease or a disorder, or ameliorating a condition associated with an abnormal or normal activity of BCKDH in a subject, comprising administering to the subject the pharmaceutical composition or the compound of the invention, thereby preventing or treating the disease and/or the disorder. In some embodiments, there is a method for preventing or treating a disease or a disorder comprising cardiovascular disease, a metabolic disorder or both.

In some embodiments, the abnormal BCKDH activity comprises reduced BCKDH activity. In some embodiments, the reduced BCKDH activity is associated with a mutant PP2Cm. In some embodiments, mutant PP2Cm comprises at least one mutation in one of its subunit genes comprising BCKDH complex, including BCKDHA (E1α subunit), BCKDHB (E1β subunit), DBT (E2 subunit), and DLD gene (E3 subunit). Non-limiting exemplary mutations are K252N/E2, R252H/E1α, and others.

In some embodiments, the disease or the disorder cardiovascular disease comprises heart failure, congestive heart failure, acute heart failure, coronary heart disease, cardiac hypertrophy, peripheral vascular disease, renovascular disease, pulmonary hypertension, vasculitis, and acute coronary syndrome or any combination thereof.

In some embodiments, the metabolic disease comprises maple syrup urine disease, nonalcoholic fatty liver disease, nonalcoholic steatohepatitis, hepatic lipid storage, muscle lipid accumulation, insulin resistance, and Type II diabetes mellitus or any combination thereof.

In some embodiments, the disease or the disorder comprises insulin resistance, impaired glucose metabolism, skin and connective tissue disorders, foot ulcerations and ulcerative colitis, endothelial dysfunction and impaired vascular compliance, hyper apo B lipoproteinemia, or any combination thereof.

In some embodiments, the disease or the disorder is or comprises a neurodegenerative disease. In some embodiments, the neurodegenerative disease is or comprises Alzheimer's disease, multiple sclerosis, dementia, Parkinson's disease, or any combination thereof.

In another aspect, there provided herein is a method for treating and/or ameliorating a condition associated with maple syrup urine disease.

In some embodiments, the method of the invention comprises administering the pharmaceutical composition of the invention to a subject, thereby selectively inhibiting BDK within the subject. In some embodiments, the method of the invention comprises administering the pharmaceutical composition of the invention to a subject, thereby enhancing BCKDH activity. In some embodiments, the method of the invention comprises administering the pharmaceutical composition of the invention to a subject, thereby enhancing BCAA catabolism within the subject. In some embodiments, the pharmaceutical composition of the invention comprises a BDK inhibitor.

In some embodiments, administering is via any one of: oral, rectal, vaginal, topical, sublingual, buccal, nasal, ophthalmic, transdermal, subcutaneous, intramuscular, intraperitoneal, intrathecal, or intravenous routes of administration, including any combination thereof.

In some embodiments, the method comprises administering the pharmaceutical composition of the invention at least 1 time, at least 2 times, at least 3 times, at least 4 times, at least 5 times, at least 7 times, or at least 10 times per day, or any value and range therebetween. Each possibility represents a separate embodiment of the invention. In some embodiments, the method comprises administering the composition or the combination of the invention 1-2 times per day or per week or per month, 1-3 times per day or per week or per month, 1-4 times per day or per week or per month, 1-5 times per day, 1-7 times per day or per week or per month, 2-3 times per day or per week or per month, 2-4 times per day or per week or per month, 2-5 times per day or per week or per month, 3-4 times per day or per week or per month, 3-5 times per day or per week or per month, or 5-7 times per day or per week or per month. Each possibility represents a separate embodiment of the invention.

In some embodiments, the method comprises administering the pharmaceutical composition of the invention to the subject at a daily or weekly or monthly dosage of 0.05 to 20 mg/kg, 0.05 to 0.1 mg/kg, 0.1 to 0.3 mg/kg, 0.3 to 0.5 mg/kg, 0.5 to 0.8 mg/kg, 0.8 to 1 mg/kg, 1 to 2 mg/kg, 2 to 5 mg/kg, 5 to 10 mg/kg, 10 to 15 mg/kg, 15 to 20 mg/kg including any range or value therebetween.

In some embodiments, the method comprises administering the pharmaceutical composition of the invention to the subject at a daily dosage (e.g. once, twice or tree-times a day) of 0.05 to 50 mg/kg, 0.05 to 0.1 mg/kg, 0.1 to 0.3 mg/kg, 0.3 to 0.5 mg/kg, 0.5 to 0.8 mg/kg, 0.8 to 1 mg/kg, 0.8 to 25 mg/kg, 0.8 to 3 mg/kg, 0.8 to 10 mg/kg, 0.8 to 15 mg/kg, 0.8 to 5 mg/kg, 3 to 5 mg/kg, 3 to 10 mg/kg, 2 to 10 mg/kg, 1 to 2 mg/kg, 2 to 5 mg/kg, 5 to 10 mg/kg, 10 to 15 mg/kg, 15 to 20 mg/kg including any range or value therebetween. In some embodiments, the daily dose can be extrapolated from the in-vivo data, such as the results presented in Examples section (e.g. Example 4).

It should be apparent to one skilled in the art, that for example in-vitro and in-vivo assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

In some embodiments, the subject is a mammal. In some embodiments, the subject is a lab animal. In some embodiments, the subject is a pet. In some embodiments, the subject is a rodent. In some embodiments, the subject is a farm animal. In some embodiments, the subject is a human subject.

In some embodiments, the subject is afflicted with a disease or disorder associated with abnormal BCAA concentration. In some embodiments, the subject is afflicted with a disease or disorder associated with reduced BCKDH activity. In some embodiments, the subject is afflicted with a disease or disorder associated with reduced BCAA catabolism. In some embodiments, the disease or disorder is as described herein.

In some embodiments, the subject comprises at least one mutation of PP2Cm.

In some embodiments, the composition of the present invention is administered in a therapeutically safe and effective amount. As used herein, the term “safe and effective amount” refers to the quantity of a component which is sufficient to yield a desired therapeutic response without undue adverse side effects, including but not limited to toxicity, such as calcemic toxicity, irritation, or allergic response, commensurate with a reasonable benefit/risk ratio when used in the presently described manner. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21st Ed., Lippincott Williams & Wilkins, Philadelphia, Pa., (2005).

In some embodiments, the effective amount or dose of the active ingredient can be estimated initially from in vitro assays. In one embodiment, a dose can be formulated in animal models and such information can be used to determine useful doses more accurately in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages may vary depending on the dosage form employed and the route of administration utilized. In one embodiment, the exact formulation, route of administration and dosage can be chosen by the individual physician in view of the patient's condition. [See e.g., Goodman and Gilman's The Pharmacological Basis of Therapeutics, 13th Ed., McGraw-Hill/Education, New York, NY (2017)].

In some embodiments, enhancing BCAA catabolism comprises at least 1%, at least 5%, at least 10%, at least 20%, at least 30%, at least 40%, at least 50%, at least 60%, at least 70%, at least 80%, at least 85%, at least 90%, at least 92%, at least 95%, at least 97%, at least 100%, at least 200%, at least 1000%, at least 10000% enhancement of BCKDH activity, including any value therebetween, as compared to a control.

In some embodiments, enhancing BCAA catabolism comprises at least 1%, at least 5%, at least 10%, at least 20%, at least 30%, at least 40%, at least 50%, at least 60%, at least 70%, at least 80%, at least 85%, at least 90%, at least 92%, at least 95%, at least 97%, at least 100%, at least 200%, at least 1000%, at least 10000% enhancement of BCAA catabolism, including any value therebetween, as compared to a control (e.g. untreated subject, as described herein). In some embodiments, enhancing BCAA catabolism comprises reducing enhanced or elevated concentration of at least one BCAA within the subject, as compared to a control. In some embodiments, reducing comprises at least 20%, 30%, 40%, 50%, at least 60%, at least 70%, at least 80%, at least 85%, at least 90%, at least 92%, at least 95%, at least 97%, at least 100%, at least 200%, at least 1000%, at least 10000% reduction of concentration of at least one BCAA within the subject, including any value therebetween.

In some embodiments, the control comprises an untreated subject. In some embodiments, the control comprises an untreated subject afflicted with a disease or disorder associated with abnormal BCAA concentration. In some embodiments, the control comprises an untreated subject having abnormal BCKDH activity. In some embodiments, abnormal BCKDH comprises reduced BCKDH activity by at least 2 times, at least 5 times at least 10 times, or more including any range between, compared to a healthy individual with normal BCKDH activity.

In some embodiments, reducing comprises at least 20%, 30%, 40%, 50%, at least 60%, at least 70%, at least 80%, at least 85%, at least 90%, at least 92%, at least 95%, at least 97%, at least 100%, at least 200%, at least 1000%, at least 10000% reduction of BDK activity, including any value therebetween.

In some embodiments, the compound of the invention has IC₅₀ in inhibiting BDK activity between 0.1 and 1 nM, between 1 and 5 nM, between 5 and 10 nM, between 10 and 50 nM, between 50 and 100 nM, between 100 and 500 nM, between 500 and 1 uM, between 1 and 5 uM, between 5 and 10 uM, including any value therebetween.

In some embodiments, the method of the invention is for preventing or treating a disease or a disorder associated with increased BCAAs concentration within the subject, comprising administering to the subject the pharmaceutical composition of the invention, thereby preventing or treating said disease or said disorder. In some embodiments, the disease or disorder is as described herein.

In some embodiments, increased BCAAs concentration comprises a concentration increase of at least one BCAA by at least 10%, at least 20%, at least 30%, at least 40%, at least 50%, at least 60%, at least 70%, at least 80%, at least 85%, at least 90%, at least 92%, at least 95%, at least 97%, at least 100%, at least 200%, at least 1000%, at least 10000%, as compared to a healthy subject, including any range or value therebetween.

In some embodiments, increased BCAAs concentration within the subject refers to a concentration of at least one BCAA within a bodily fluid (e.g. blood, urine, etc.) and/or a tissue of the subject.

In some embodiments, the method further comprising a step preceding said administering, comprising determining concentration of at least one BCAA in said subject, wherein an increased concentration of the at least one BCAA in said subject, is indicative of said subject being suitable for said treating. In some embodiments, the step preceding said administering, comprising determining BCKDH activity in said subject, wherein reduced BCKDH activity in said subject, is indicative of said subject being suitable for the treating.

In some embodiments, the step preceding said administering, comprising determining BDK inhibitory activity of the compound or the pharmaceutical composition of the invention. In some embodiments, determining BDK inhibitory activity is according to an assay described herein (Example 4).

In some embodiments, determining is in a sample obtained or derived from the subject. In some embodiments, determining BCKDH activity in the subject according to an assay described herein (Example 4). In some embodiments, a subject suitable for the treatment is as described herein.

In some embodiments, a subject suitable for the treatment has at least one mutation within the PP2Cm gene. In some embodiments, at least one mutation is within a BCKDH subunit of the PP2Cm gene (such as K252N/E2 mutation). In some embodiments, a subject suitable for the treatment is selected from iMSUD subject, thiamine-responsive subject, and classic MSUD subject with minor residual activity. In some embodiments, intermediate and intermittent MSUD (=iMSUD) subjects have between 5 and 70%, between 5 and 10%, between 10 and 20%, between 20 and 50%, between 50 and 70%, of normal BCKDH activity including any range between.

### Definitions

As used herein, the term “alkyl” describes an aliphatic hydrocarbon including straight chain and branched chain groups. In some embodiments, the alkyl group has 1 to 20 carbon atoms, between 1 and 10, between 1 and 5, between 5 and 10, between 10 and 15, between 15 and 20, including any range between.

In some embodiments, the alkyl group has 21 to 100 carbon atoms, and more preferably 21-50 carbon atoms. Whenever a numerical range e.g., “21-100”, is stated herein, it implies that the group, in this case the alkyl group, may contain 21 carbon atom, 22 carbon atoms, 23 carbon atoms, etc., up to and including 100 carbon atoms. In the context of the present invention, a “long alkyl” is an alkyl having at least 20 carbon atoms in its main chain (the longest path of continuous covalently attached atoms). A short alkyl therefore has 20 or less main-chain carbons. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein. Additionally, the term “cycloalkyl” further encompasses a heterocyclyl ring, as described herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine, or iodine.

The term “haloalkyl” describes an alkyl group as defined herein, further substituted by one or more halide(s).

The term “haloalkoxy” describes an alkoxy group as defined herein, further substituted by one or more halide(s).

The term “hydroxyl” or “hydroxy” describes a —OH group.

The term “mercapto” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amino” describes a —NR′R″ group, with R′ and R″ as described herein.

The term “heterocyclyl” describes a monocyclic or fused ring group having in the ring(s) one or more atoms such as nitrogen, oxygen, and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” or “carboxylate” describes a —C(O)OR′ group, where R′ is hydrogen, alkyl, cycloalkyl, alkenyl, aryl, heteroaryl (bonded through a ring carbon) or heterocyclyl (bonded through a ring carbon) as defined herein.

The term “carbonyl” describes a —C(O)R′ group, where R′ is as defined hereinabove.

The above-terms also encompass thio-derivatives thereof (thiocarboxy and thiocarbonyl).

The term “thiocarbonyl” describes a —C(S)R′ group, where R′ is as defined hereinabove.

A “thiocarboxy” group describes a —C(S)OR′ group, where R′ is as defined herein.

A “sulfinyl” group describes an —S(O)R′ group, where R′ is as defined herein.

A “sulfonyl” or “sulfonate” group describes an —S(O)2R′ group, where R′ is as defined herein.

A “carbamyl” or “carbamate” group describes an —OC(0)NR′R″ group, where R′ is as defined herein and R″ is as defined for R′.

A “nitro” group refers to a —NO2 group.

The term “amide” as used herein encompasses C-amide and N-amide.

The term “C-amide” describes a —C(O)NR′R″ end group or a —C(O)NR′— linking group, as these phrases are defined hereinabove, where R′ and R″ are as defined herein.

The term “N-amide” describes a —NR″C(O)R′ end group or a —NR′C(O)— linking group, as these phrases are defined hereinabove, where R′ and R″ are as defined herein.

The term “carboxylic acid derivative” as used herein encompasses carboxy, amide, carbonyl, anhydride, carbonate ester, and carbamate.

A “cyano” or “nitrile” group refers to a —CN group.

The term “azo” or “diazo” describes an —N═NR′ end group or an —N═N— linking group, as these phrases are defined hereinabove, with R′ as defined hereinabove.

The term “guanidine” describes a —R′NC(N)NR″R″′ end group or a —R′NC(N) NR″— linking group, as these phrases are defined hereinabove, where R′, R″ and R″′ are as defined herein.

As used herein, the term “azide” refers to a —N3 group.

The term “sulfonamide” refers to a —S(O)2NR′R″ group, with R′ and R″ as defined herein.

The term “phosphonyl” or “phosphonate” describes an —OP(O)—(OR′)2 group, with R′ as defined hereinabove.

The term “phosphinyl” describes a —PR′R″ group, with R′ and R″ as defined hereinabove.

The term “alkylaryl” describes an alkyl, as defined herein, which substituted by an aryl, as described herein. An exemplary alkylaryl is benzyl.

The term “heteroaryl” describes a monocyclic (e.g. C5-C6 heteroaryl ring) or fused ring (i.e. rings which share an adjacent pair of atoms) group having in the ring(s) one or more atoms, such as, for example, nitrogen, oxygen, and sulfur and, in addition, having a completely conjugated pi-electron system. In some embodiments, the terms “heteroaryl” and “C5-C6 heteroaryl” are used herein interchangeably. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazol, pyridine, pyrrole, oxazole, indole, purine, and the like.

As used herein, the terms “halo” and “halide”, which are referred to herein interchangeably, describe an atom of a halogen, that is fluorine, chlorine, bromine, or iodine, also referred to herein as fluoride, chloride, bromide, and iodide.

The term “haloalkyl” describes an alkyl group as defined above, further substituted by one or more halide(s).

### General

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described active ingredients prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of inflammatory disorders.

The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression.

Conversely, the term “treatment” refers to the clinical application of active agents to combat an already existing condition whose clinical presentation has already been realized in a patient.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising” indicate the inclusion of any recited integer or group of integers but not the exclusion of any other integer or group of integers.

As used herein, the term “consists essentially of” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure, or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises” “comprising”, and “having” are/is interchangeable with “consisting”.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent, or patent application was specifically and individually indicated to be incorporated herein by reference. In addition, citation, or identification of any reference in this application shall not be construed as an admission that such reference is available as prior art to the present invention. To the extent that section headings are used, they should not be construed as necessarily limiting.

### EXAMPLES

Generally, the nomenclature used herein, and the laboratory procedures utilized in the present invention include molecular, biochemical, and microbiological techniques. Such techniques are thoroughly explained in the literature. Biochemical methods for potency determination:

Cell-free BDK activity assay was developed based on protocols and published work of David Chuang as described in Tso et al. 2014. Briefly, The main components of the enzymatic cell-free activity assay are the kinase-BDK (MBP-BDK), its 14 kDa synthetic substrate Lip-LBD-PhP, and ATP. The detection is being done with Promega ADP-Glo™ kinase kit. The assay is robust generating an assay window (AW)>5 and Z′−factor>0.5. The concentrations of all assay components were reduced relative to published concentrations and the assay was validated using known BDK inhibitors. Additionally, similar activity of the rat and human enzymes was demonstrated, confirming the high homology between both proteins.

Constructs: a synthetic 14 KDa peptide that serves as a substrate for BDK instead of the multimeric BCKDH. This peptide, named LBD-PhP, is composed of the lipoyl binding domain (LBD) of the BCKDH E2 subunit connected by a linker to the phosphorylation peptide (PhP) of the E1α subunit. LBD-PhP is lipoylated in vitro using the E. Coli enzyme—lipoylate ligase (Lp1A) to achieve enhancement of BDK substrate phosphorylation. Additionally, a chimeric BDK in which the kinase is fused in its N-terminus to MBP, generating-MBP-BDK to increase BDK solubility and stability.

Reaction: The ADP detection is composed of the following steps:

Kinase reaction is performed by adding BDK, lip-LBD-PhP and ATP and incubating for 60 min at room temperature. This is done in solid white 384-well plates and the total volume of the reaction is 10 μl.

ADP-Glo reagent is added to terminate the kinase reaction and to deplete the remaining ATP and incubated for 40 min.

Kinase detection reagent is added for ADP detection: The ADP is converted to ATP and using a series of enzymatic reactions the newly synthesized ATP generates a luminescent signal which is proportional to the amount of the initial ADP formed by the kinase reaction. Luminescence is measured using a plate reader (Tecan Spark 20M plate reader).

In a typical experiment the AW and Z-factor are calculated from 12 repeats of positive control (100% activity) and 12 repeats of negative control (0% activity).

Positive control (100% activity)=maximal kinase activity with ATP and substrate. Negative control (0% activity)=minimal kinase activity with ATP alone (no substrate). AW: Average_{100%}/Average_{0%}. Z-factor: 1−((3*STDEV_{100%}+3*STDEV_{0%})/(Average_{100%}−Average_{0%})).

Each 384-well plate-based screening experiment includes serial dilutions of 14 different compounds and the following controls: 1. A known BDK tool compound BT2 (for allosteric inhibitors)/Radicicol (for ATP-pocket inhibitors) 2. Twelve repeats of positive and negative controls. Analyzed compounds and tool compounds are serially diluted, 8-point half log from 50 μM to 16 nM in triplicates. The positive and negative controls are used to calculate AW and Z-factor and to enable data normalization.

### Example 1

### In-Vitro BDK Inhibitors

Several compounds of the invention showed significant BDK inhibition in-vitro, showing micromolar and sub-micromolar IC₅₀ values. Some compound exhibited IC₅₀ values between 100 and 500 nM (e.g. in a cell-free biochemical assay). Exemplary BDK inhibitors are represented below.

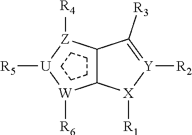

| Name | X | Y | U | W | Z | R1 | R2 | R3 | R4 | R5 | R6 |
|---|---|---|---|---|---|---|---|---|---|---|---|
| RMO-149 | S | C | C | S | C | — | COOH | H | H | Cl | H |
| RMO-176 | S | C | C | N | S | H | COOH | H | — | N(Et)₂ | H |
| RMO-189 | S | C | C | N | N | — | COOH | H | Me | H | H |
| RMO-140 | N | C | C | C | O | Me | COOH | H | — | H | H |
| RMO-141 | N | C | C | C | O | H | COOH | H | — | H | H |
| RMO-142 | N | C | C | C | O | H | COOH | H | — | Me | H |
| RMO-181 | N | C | C | S | C | H | COOH | H | — | Br | H |
| RMO-173 | N | C | C | S | N | H | COOH | H | — | Br | H |
| RMO-229 | N | N | C | S | C | Ph | — | Ph | Cl | COOH | H |

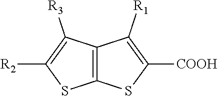

| Name | R1 | R2 | R3 |
|---|---|---|---|
| RMO-149 | H | Cl | H |
| RMO-179 | H | Br | H |
| RMO-177 | H | C(O)OMe | H |
| RMO-178 | H | SO₂NH₂ | H |
| RMO-182 | H | H | H |
| RMO-208 | H | H | COOH |
| RMO-195 | Cl | H | H |
| RMO-163 | Cl | Cl | H |
| RMO-198 | Me | Me | COOH |
| RMO-197 | Me | C(O)OMe | Me |
| RMO-201 | H | 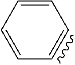 | |
| RMO-800 | H | F | F |
| RMO-801 | F | F | H |
| RMO-1153 | F | H | H |
| RMO-1153B | H | F | H |

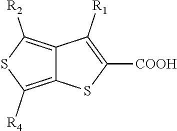

| Name | R1 | R2 | R4 |
|---|---|---|---|
| RMO-1149 | H | F | F |
| RMO-1150 | F | H | F |
| RMO-1154 | F | H | H |
| RMO-832 | H | H | H |
| RMO-831 | H | Methyl | Methyl |
| RMO-812 | H | Br | Br |

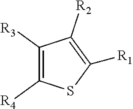

| Name | R1 | R2 | R3 | R4 |
|---|---|---|---|---|
| RMO-235 | COOH | H | H | Ph |
| RMO-233 | COOH | H | H | 4-Cl—Ph |
| RMO-231 | COOH | H | H | 3-Cl—Ph |
| RMO-238 | COOH | H | H | O—Ph |
| RMO-828 | COOH | H | H | 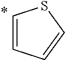 |
| RMO-232 | COOH | H | Ph | H |
| RMO-230 | CH₂COOH | Me | H | H |
| RMO-148 | CH₂OH | S—Ph | COOH | Me |

Furthermore, compound RMO-1152:

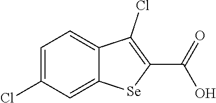 [SMILES: O=C(O)c1[se]c2cc(Cl)ccc2c1Cl]

has been successfully synthesized according to a procedure described in Example 2 below. RMO-1152 exhibited BDK inhibitory activity in-vitro, with IC50 values of between 2.86 and 13.6 in lymphoblasts cells.

Fluorinated compounds (such as RMO 800, 801, 1149, 1150, 1153, 1153B, 1154) showed enhanced activity in-vitro, with IC50 values of between 100 and 800 nM.

### Example 2

### Synthetic Procedures

### General Procedure for Preparation of Compound 163_2

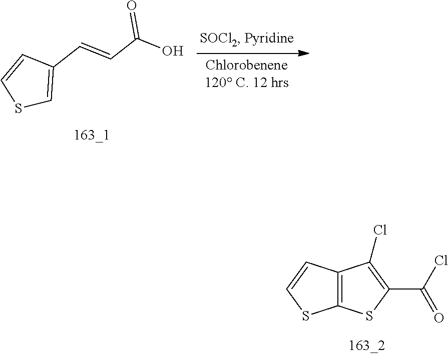 [SMILES: O=C(O)/C=C/c1ccsc1 ; O=C(Cl)c1sc2sccc2c1Cl]

A mixture of compound 163_1 (4.00 g, 25.9 mmol, 1.00 eq), Pyridine (205 mg, 2.59 mmol, 210 uL, 0.10 eq), SOC12 (15.4 g, 130 mmol, 9.41 mL, 5.00 eq) in Chlorobenzene (32.0 mL) was stirred at 120° C. for 12 hrs. TLC (Petroleum ether: Ethyl acetate=5:1) indicated Reactant 1 (Rf=0.05) was consumed completely and many new spots formed. The reaction mixture was concentrated under reduced pressure at 40° C. to remove solvent. The residue was purified by column chromatography (SiO2, petroleum ether) to get Compound 163_2 (0.50 g, 911 umol, 3.51% yield, 43.2% purity) as a white solid, which was confirmed by LCMS.

### General Procedure for Preparation of Compound 163_3

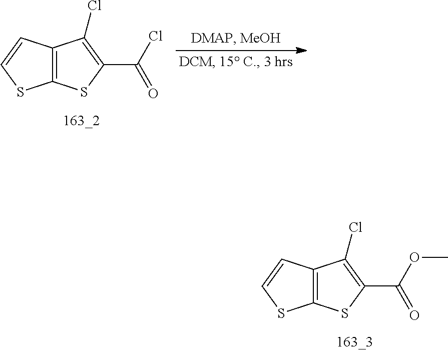

A mixture of DMAP (247 mg, 2.02 mmol, 1.20 eq), MeOH (64.9 mg, 2.02 mmol, 81.9 uL, 1.20 eq) in DCM (6.00 mL) was added compound 163_2 (400 mg, 1.69 mmol, 1.00 eq) drop-wise at 0° C. The reaction mixture was stirred at 15° C. for 3 hrs. TLC (Petroleum ether: Ethyl acetate=5:1) indicated Reactant 1 (Rf=0.5) was consumed completely and one new spot formed (Rf=0.55). The reaction mixture was concentrated under reduced pressure at 40° C. to remove solvent. The residue was purified by prep-TLC (SiO₂, Petroleum ether: Ethyl acetate=4:1). Compound 163_3 (0.93 g, 3.38 mmol, 80.1% yield, 84.5% purity) was obtained as a white solid, which was confirmed by LCMS (RT=0.686 min, m/z=233.2) and HNMR.

### General Procedure for Preparation of Compound RMO-165:

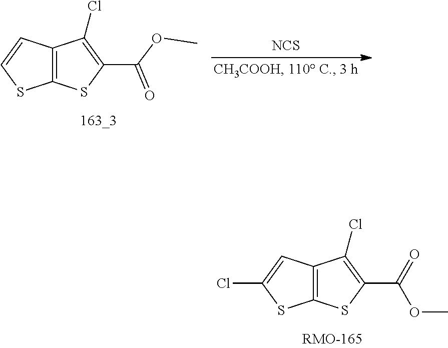

To a solution of CH₃COOH (5.25 g, 87.4 mmol, 5.00 mL, 203 eq) was added 163_3 (100 mg, 429 umol, 1.00 eq) and NCS (68.9 mg, 516 umol, 1.20 eq) at 25° C., then heated to 110° C. The mixture was stirred at this temperature for 3 hrs. TLC (Petroleum ether: Ethyl acetate=8:1) indicated 163_3 (Rf=0.56) was consumed completely, and one new spot formed. The reaction mixture was poured into saturated NaHCO₃ solution (100 mL) to adjusted pH=7˜8, then extracted with ethyl acetate (30 mL*3). The combined organic layers were washed with brine (30 mL*3), dried over Na₂SO₄, filtered, and concentrated under reduced pressure to give a residue. The residue was purified by prep-TLC (SiO₂, Petroleum ether:Ethyl acetate=8:1). Compound RMO-165 (76 mg, 97.2% purity) was obtained as a white solid, which was confirmed by LCMS, HNMR, HSQC and HMBC and GCMS (RT=6.858 min, m/z=265.90).

### General Procedure for Preparation of Compound RMO-163

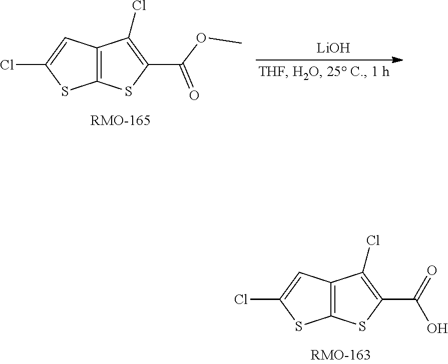

To a solution of RMO-165 (20.0 mg, 74.9 umol, 1.00 eq) in THF (2.00 mL) and H₂O (0.10 mL) was added LiOH (17.9 mg, 749 umol, 10.0 eq). The mixture was stirred at 25° C. for 1 hr. LC-MS showed Reactant 1 was consumed and one main peak with desired m/z was detected. The mixture was concentrated to give a residue. The residue was purified by pre-HPLC (column: Phenomenex Synergi C18 150*25 mm*10 um; mobile phase: [water (0.1% TFA)-ACN]; B %: 42%-72%, 10 min). Compound RMO-163 (6.89 mg, 37.6 umol, 50.2% yield, 95.2% purity) was obtained as a white solid which was confirmed by HNMR and LCMS and HPLC.

General Procedure for Preparation of Compound RMO-1152 [3,6-dichlorobenzo[b]selenophene-2-carboxylic acid]

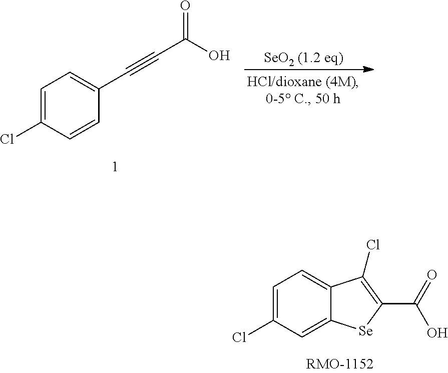 [SMILES: O=C(O)C#Cc1ccc(Cl)cc1 ; "SeO2 (1.2 eq)" ; O=C(O)c1[se]c2cc(Cl)ccc2c1Cl]

To a suspension of 3-(4-chlorophenyl)prop-2-ynoic acid (1.00 g, 5.54 mmol, 1.0 eq) in HCl/dioxane (20.0 mL, 4 M) was added SeO2 (737.3 mg, 6.64 mmol, 1.2 eq) at 0° C. The mixture was stirred at 5° C. for 45 h. LC-MS showed the starting material was consumed and desired MS (248.6, [M-CO2-H]-, ESI-) was detected. The reaction mixture was concentrated in reduced pressure to give a residue. The residue was dissolved with methanol (5.00 mL) and purified by Prep-HPLC (column: Waters Xbridge 150*25 mm*5 um; mobile phase: [water (0.05% ammonia hydroxide v/v)-ACN]; ACN %: 5%-35%, 10 min) and lyophilized to give a crude product as a white solid. LC-MS indicated that it was not pure.

The crude product was purified again by Prep-HPLC (column: Phenomenex Luna C18 75*30 mm*3 um; mobile phase: [water (0.1% TFA)-ACN]; ACN %: 48%-78%, 7 min) and lyophilized to give 3,6-dichlorobenzo[b]selenophene-2-carboxylic acid (93.90 mg, 315.08 umol, 5.69% yield, 98.64% purity) as a white solid. The structure was confirmed by 1H NMR and 2D NMR.

General Procedure for Preparation of Compound RMO-800, [4,5-difluorothieno[2,3-b]thiophene-2-carboxylic acid].

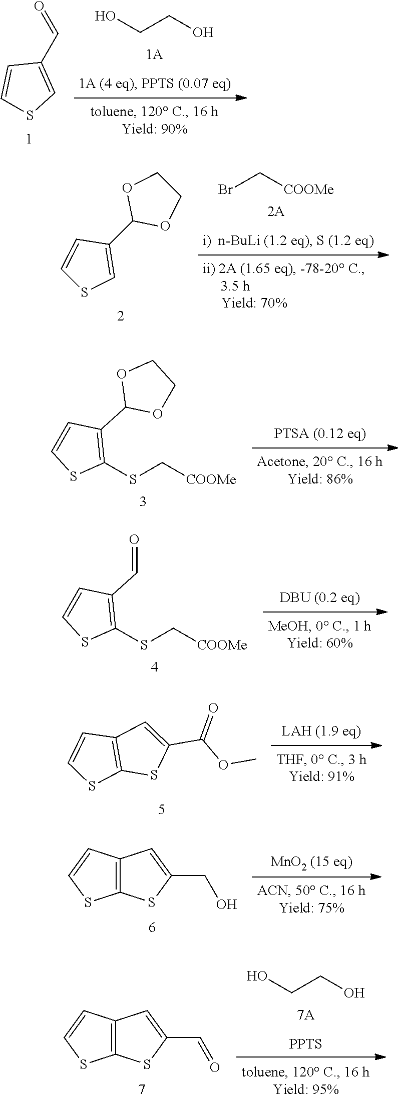

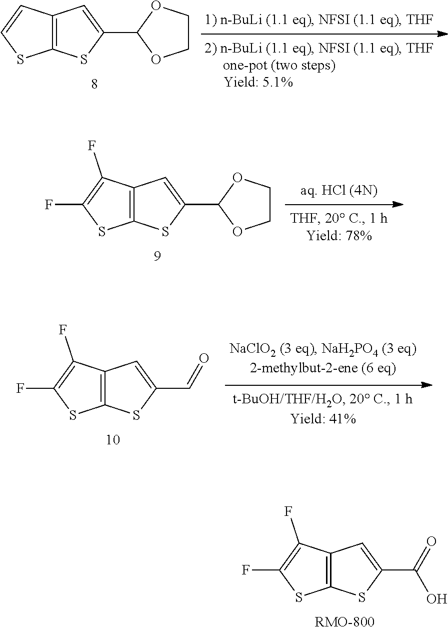 [SMILES: c1cc2cc(C3OCCO3)sc2s1 ; Fc1sc2sc(C3OCCO3)cc2c1F ; O=Cc1cc2c(F)c(F)sc2s1 ; O=C(O)c1cc2c(F)c(F)sc2s1]

General Procedure for Preparation of Compound RMO-801 [2,4-difluorothieno[2,3-b]thiophene-5-carboxylic acid]:

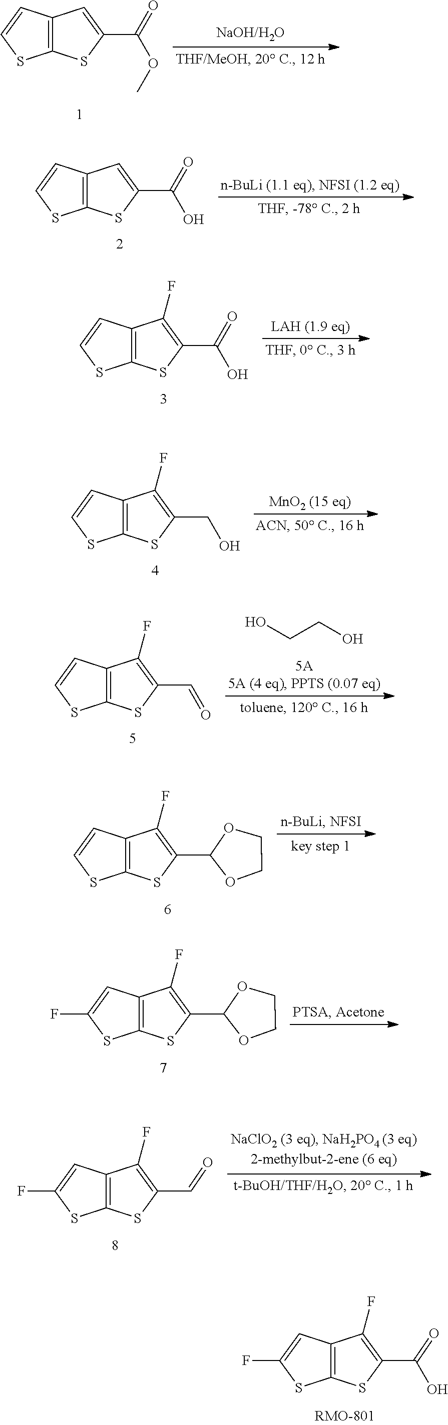

General Procedure for Preparation of Compound RMO-1149, [4,6-difluorothieno[3,4-b]thiophene-2-carboxylic acid]:

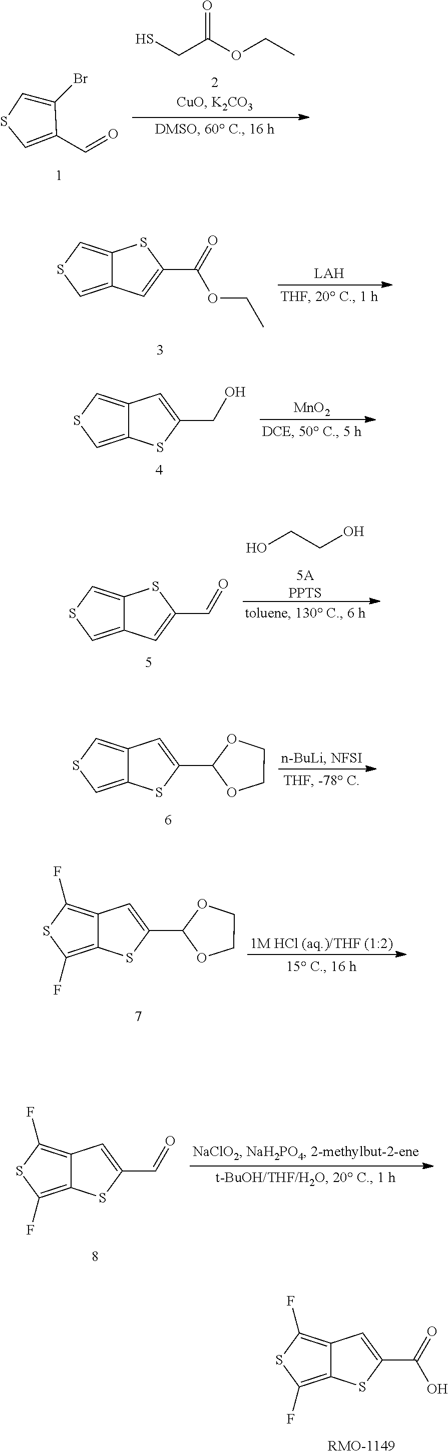

General Procedure for Preparation of Compound RMO-1150, [3,6-difluorothieno[3,4-b]thiophene-2-carboxylic acid]:

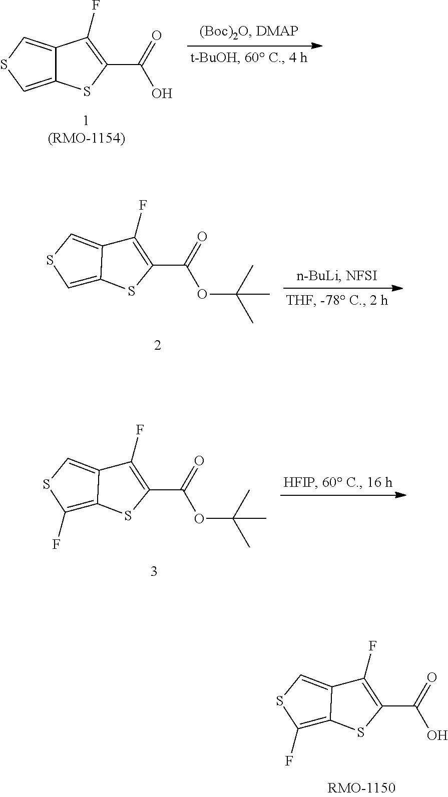 [SMILES: O=C(O)c1sc2cscc2c1F ; CC(C)(C)OC(=O)c1sc2cscc2c1F ; CC(C)(C)OC(=O)c1sc2c(F)scc2c1F ; O=C(O)c1sc2c(F)scc2c1F]

General Procedure for Preparation of Compound RMO-1153, [4-fluorothieno[2,3-b]thiophene-5-carboxylic acid]:

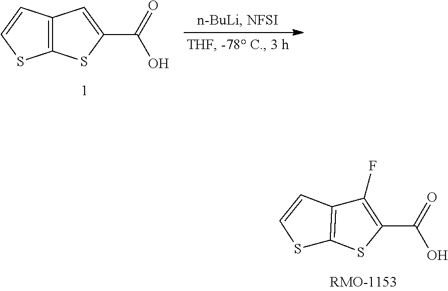 [SMILES: O=C(O)c1cc2ccsc2s1 ; O=C(O)c1sc2sccc2c1F]

General Procedure for Preparation of Compound RMO-1153B [5-fluorothieno[2,3-b]thiophene-2-carboxylic acid]:

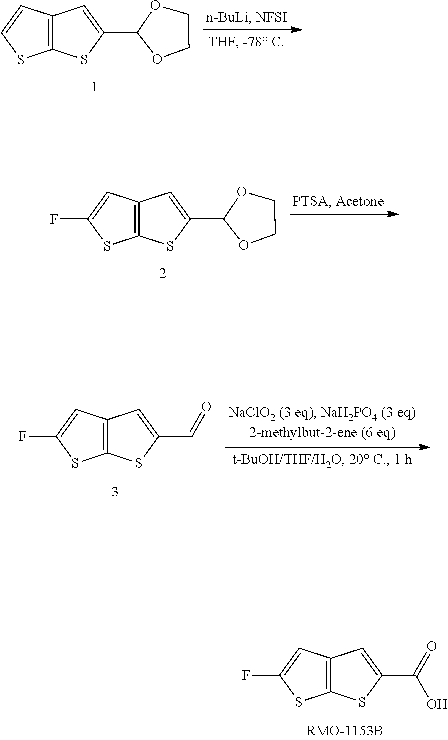 [SMILES: c1cc2cc(C3OCCO3)sc2s1 ; Fc1cc2cc(C3OCCO3)sc2s1 ; O=Cc1cc2cc(F)sc2s1 ; O=C(O)c1cc2cc(F)sc2s1]

General Procedure for Preparation of Compound RMO-1154, [3-fluorothieno[3,4-b]thiophene-2-carboxylic acid]:

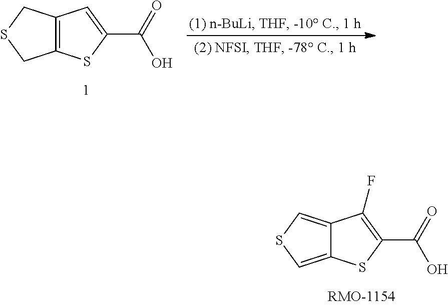 [SMILES: O=C(O)c1cc2c(s1)CSC2 ; O=C(O)c1sc2cscc2c1F]

### Example 3

### In-Vivo And Ex-Vivo Studies

Exemplary compounds of the invention have been tested in biochemically (e.g., according to the cell-free BDK activity assay, described hereinabove) and in a cell culture (human lymphoblasts), to determine BDK inhibitory activity thereof. The results showing IC50 of the tested compound are shown in Table 1 below.

TABLE 1

| | IC50 | IC50 |
|---|---|---|
| | BDK | lymphoblasts |
| Name | (nM) | (nM) |
| RMO-800 | 100 | 100 |
| RMO-801 | 470 | 170 |
| RMO-1149 | 800 | 380 |
| RMO-1153 | 490 | 290 |
| RMO-1153B | 610 | |
| RMO-1154 | 560 | 580 |

Additionally, compound

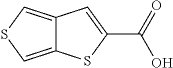 [SMILES: O=C(O)c1cc2cscc2s1]

showed significant BDK inhibitory activity in-vitro.

Various pharmacokinetic parameters of exemplary compounds of the invention have been established in-vivo and are presented in Table 2 below.

TABLE 2

| | Plasma | Volume of | Oral bio- | |
|---|---|---|---|---|
| | protein | distribution | availability | Exposure |
| Name | binding | (V_{d}) | (F) | (AUC) |
| RMO-800 | 98.8% | 0.16 L/kg | NA | 3584 h · ng/mL |
| (IV) | (mouse) | | | (1 mg/kg) |
| RMO-800 | 99.2% | NA | 136% | 14742 h · ng/mL |
| (IP) | (human) | | | (3 mg/kg) |
| RMO-800 | | NA | 118% | 12669 h · ng/mL |
| (PO) | | | | (3 mg/kg) |
| RMO-1154 | 89.2% | 0.15 L/kg | NA | 6129 h · ng/mL |
| (IV) | (mouse) | | | (1 mg/kg) |
| RMO-1154 | 98.5% | NA | 98% | 17977 h · ng/mL |
| (IP) | (human) | | | (3 mg/kg) |
| RMO-1154 | | NA | 104% | 19436 h · ng/mL |
| (PO) | | | | (3 mg/kg) |

### Example 4

Efficacy of the exemplary compounds of the invention has been evaluated on cells derived from MSUD patients. It is postulated, that the assay disclosed hereinbelow, can be utilized for predicting patient compatibility/adaptivity to the treatment by the compounds disclosed herein.

A preliminary test of patient's cells with the exemplary compounds of the invention enable to support the correlation between genetic mutations and the ability to modulate the BCKDH complex.

As opposed to healthy individuals with normal BCKDH activity (also referred to as 100% BCKDH activity), intermediate and intermittent MSUD (=iMSUD) patients are have only 5-70% of normal BCKDH activity. iMSUD patients have mutations in BCKDH subunits or PP2Cm genes. To this end the inventors postulated that patients responsive to BDK inhibition treatment (by utilizing any of the compounds and/or pharmaceutical compositions disclosed herein) are selected from iMSUD patients, thiamine-responsive patients, and some classical MSUD patients.

BCKDH complex catabolizing BCAAs derivatives, BCKAs, and its activity is regulated by a kinase (BDK) which inhibits BCKDH activity, and a phosphatase (PP2Cm), which enhanced BCKDH activity. The compounds of the invention are presumed to lower BCAAs within a subject by inhibiting BDK activity, and as a consequence enhancing subject's BCKDH activity.

BDK mediated increase of the BCKDH activity by incubating human lymphoblasts upon incubation of known BDK inhibitors (BT2, BT2F, and BT3) with lymphoblasts, has been demonstrated in both WT and iMSUD patient derived cells. (see Shih-Chia Tso et al., JBC, 2014).

Accordingly, the inventors performed cell studies in order to test the efficacy of the exemplary compounds of the invention on the BCKDH E1α subunit phosphorylation level, as it is BDK substrate in WT and iMSUD lymphoblasts and fibroblasts. Efficacy of the tested compounds RMO-1154 and RMO-800 in 2 MSUD lymphoblasts lines has been determined using cell-based ELISA to quantify BDK activity in treated cells as monitored by the phosphorylation level of BDK substrate, E1α. Healthy donor cells and BT2 (commercially available BDK inhibitor) have been used as positive controls.

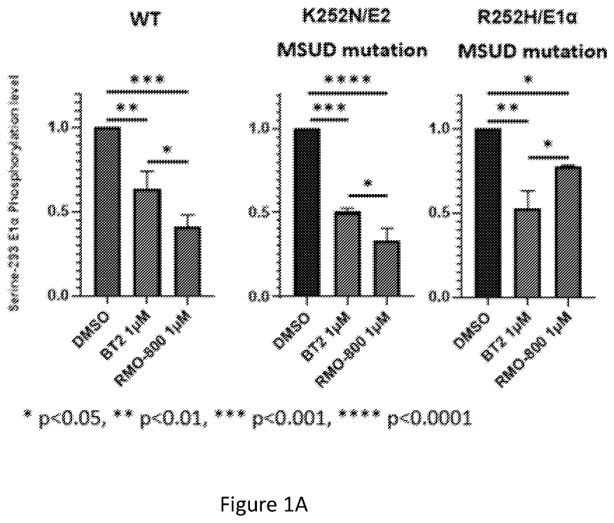

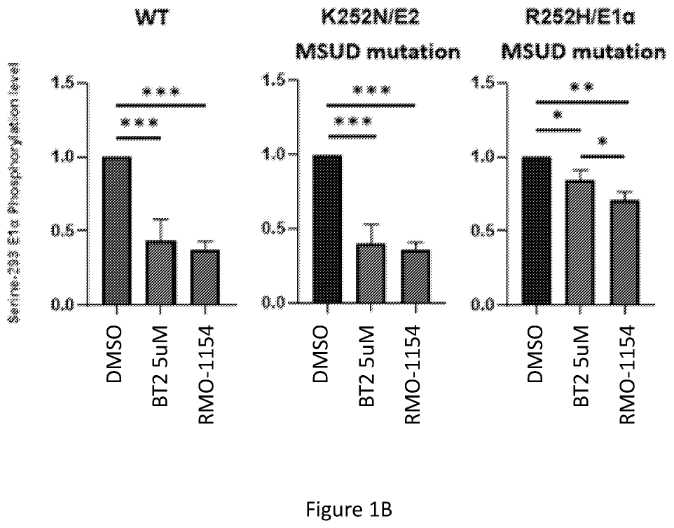

The results of these experiments are represented in FIG. 1, showing a similar or a superior effect of the tested compounds, compared to BT2. Especially, the tested compounds showed unexpected efficacy substantially reducing BDK phosphorylation (up to about 70% reduction of BDK activity, compared to untreated cells) of iMSUD patient derived lymphoblasts (K252N/E2 mutation). The results demonstrate that patients with these mutations are suitable candidates for a potential BDK inhibitor treatment.

An alternative assay for assessment of BCKDH activity comprises measuring of BCKAs blood (e.g. plasma) concentration within the subject. BCKAs (such as a-keto isovalerate (abbreviated as KIV), sodium a-keto isocaproate (abbreviated as KIC), sodium a-keto β-methylvalerate (abbreviated as KMV)) are downstream metabolites of BCAAs, and the substrates of BCKDH complex, and are considered to be reliable and sensitive biomarkers for BDK inhibition and BCKDH complex activity enhancement.

BCKA blood concentration of a subject can be determined by various analytical methods, such as by LC-MS.

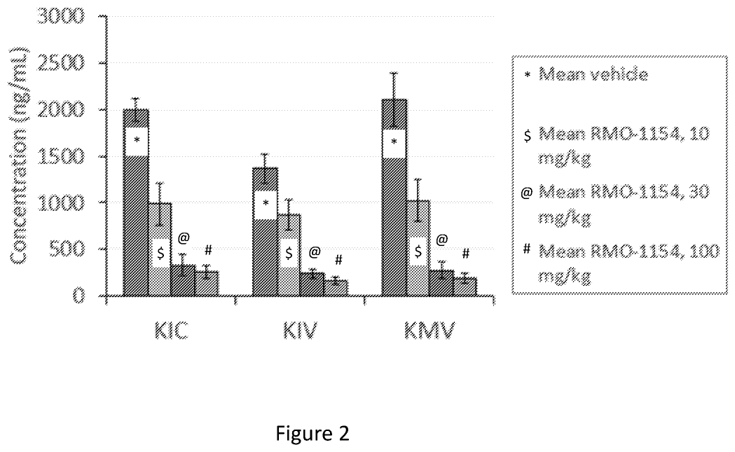

The inventors performed an in-vivo study by administering an exemplary compound of the invention (RMO-1154) to 8-10 week-old B57/C WT mice (n=5). RMO-1154 has been administered (per os, 3 single doses a day every 8 hrs, for 7 days) at various single doses (10 mg/kg, 30 mg/kg, and 100 mg/kg). At day 8, KIV, KIC and KMV plasma concentration has been determined by LC-MS. The results of this experiment are presented in FIG. 2. As shown in FIG. 2, the compound of the invention resulted in about 2-fold, 8-10-fold and 10-12-fold reduction of BCKA (such as KIC and/or KMV) plasma concentrations, when administered at 10 mg/kg, 30 mg/kg, and 100 mg/kg, respectively.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A compound including any tautomer or a salt thereof, wherein said compound is represented by Formula II: 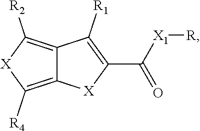
wherein:
both X represent S;
each of R1, R2, and R4 is independently H or a substituent selected from —NR′₂, —CN, —OR′, —CONR′₂, —CO₂R′, —SO₂R′, hydroxy(C₁-C₆ alkyl), F, C₁-C₆ alkyl, and C₁-C₆ haloalkyl or a combination thereof;
and at least two of R1, R2, and R4 are fluoro;
or wherein said compound is represented by Formula III: 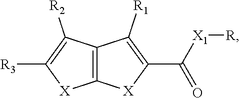
wherein:
each of R1, R2, and R3 is independently H or a substituent selected from F, optionally substituted C₁-C₆ alkyl, —NR₂, —CN, —OR, —CONR₂, —CO₂R, —SO₂R, hydroxy(C₁-C₆ alkyl), and C₁-C₆ haloalkyl or a combination thereof;
at least one X is S or Se;
at least one of R1, R2, and R4 comprises F; and
R represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl or a combination thereof.

2. (canceled)

3. (canceled)

4. (canceled)

5. (canceled)

6. (canceled)

7. (canceled)

8. The compound of claim 1, wherein R is hydrogen.

9. (canceled)

10. The compound of claim 1, wherein the compound is represented by Formula III and wherein at least two of R₁, R₂, and R₃ are fluoro; optionally wherein R is hydrogen.

11. (canceled)

12. The compound of claim 1, wherein said C₁-C₆ haloalkyl is selected from the group comprising —CF₃, —CHF₂, —CH₂F, —CH₂—CF₃, —CH₂—CHF₂, or —CH₂—CH₂F.

13. The compound of claim 1, wherein the compound is selected from: 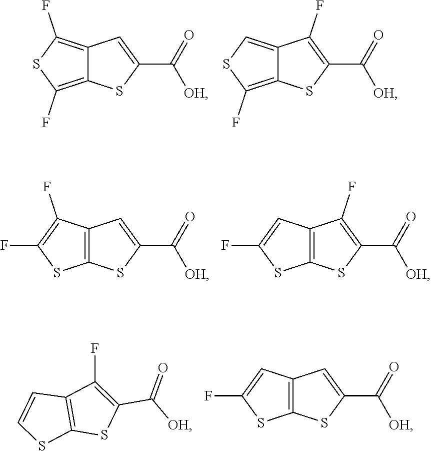
including any salt or any combination thereof.

14.-21. (canceled)

22. A method for preventing or treating a disease or a disorder associated with increased BCAAs concentration within a bodily fluid and/or a tissue of a subject, comprising administering to the subject a pharmaceutical composition comprising a compound, a tautomer thereof, a salt or a combination thereof and a pharmaceutically acceptable carrier, wherein said compound is represented by or comprises Formula 6: 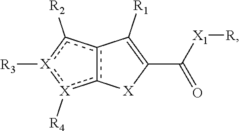
wherein:
X₁ represents S, NR′ or O; [symbol]
represents a single or a double bond;
each X is independently selected from S, Se, and CH, and at least one X is S or Se;
R₃ and R₄ each independently is absent or is selected from the group comprising hydrogen, halo, optionally substituted C₁-C₆ alkyl, and C₁-C₆ haloalkyl or a combination thereof;
R₁ represents hydrogen, or a substituent comprising halo, C₁-C₆ haloalkyl, or optionally substituted C₁-C₆ alkyl;
R₂ represents hydrogen, or is selected from the group comprising halo, —NO₂, —CN, —OH, —CONH₂, —CONR′₂, —CO₂R′, —SO₂R′, optionally substituted C₁-C₆ alkyl, —NH₂, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, or a combination thereof; or wherein R₂ and R₃ are interconnected so as to form a cyclic ring;
each R′ and R independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl or a combination thereof.

23. The method of claim 22, wherein said compound is represented by Formula 8E: 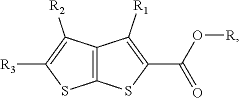
wherein:
each of R1, R2, and R3 is independently H or a substituent selected from halo, optionally substituted C₁-C₆ alkyl, —NR₂, —CN, —OR, —CONR₂, —CO₂R, —SO₂R, hydroxy(C₁-C₆ alkyl), and C₁-C₆ haloalkyl or a combination thereof; optionally wherein at least one of R1, R2, and R3 is halo.

24. (canceled)

25. The method of claim 22, wherein halo is F.

26. The method of claim 22, wherein said compound is represented by Formula 7B: 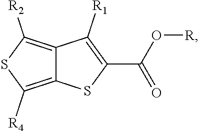
wherein:
each of R1, R2, and R4 is independently H or a substituent selected from —NR′₂, —CN, —OR′, —CONR′₂, —CO₂R′, —SO₂R′, hydroxy(C₁-C₆ alkyl), halo, C₁-C₆ alkyl, and C₁-C₆ haloalkyl or a combination thereof, optionally wherein at least one of R1, R2, and R4 is fluoro

27. The method of claim 22, wherein said compound comprises 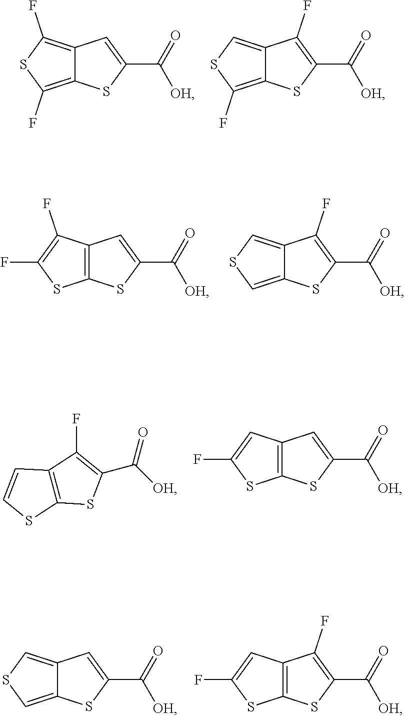
or a combination thereof.

28.-32. (canceled)

33. The method of claim 22, wherein said disease or said disorder comprises a cardiovascular disease, a metabolic disorder, a neurodegenerative disorder or any combination thereof.

34. The method of claim 33, wherein said cardiovascular disease comprises heart failure, congestive heart failure, acute heart failure, coronary heart disease, cardiac hypertrophy, peripheral vascular disease, renovascular disease, pulmonary hypertension, vasculitis, and acute coronary syndrome or any combination thereof.

35. The method of claim 33, wherein said metabolic disorder comprises maple syrup urine disease, nonalcoholic fatty liver disease, nonalcoholic steatohepatitis, hepatic lipid storage, muscle lipid accumulation, Type I diabetes, and Type II diabetes mellitus or any combination thereof.

36. The method of claim 22, wherein said increased BCAAs concentration comprises a concentration increase of at least one BCAA by at least 10%, as compared to a healthy subject.

37. The method of claim 22, further comprising a step preceding said administering, comprising (i) determining concentration of at least one BCAA in said subject; or (ii) determining BCKDH activity in said subject; wherein an increased concentration of the at least one BCAA in said subject, or a reduced BCKDH activity in said subject is indicative of said subject being suitable for said treating.

38. The method of claim 22, wherein further comprising a step preceding said administering, comprising determining BDK inhibitory activity of the compound, wherein a reduced BDK activity in said subject is indicative of said subject being suitable for said treating.

39. The method of claim 37, wherein said determining is in a sample obtained or derived from the subject.

40. The method of claim 22, wherein the disease or the disorder is associated with a mutant PP2Cm.

41. (canceled)
